(19) United States
(12) Patent Application Publication
(10) Pub. No.: US 2026/0317704 A1
(43) Pub. Date: Oct. 15, 2026

(54) VASCULAR PLANT BASED SCAFFOLD AND IN-VITRO METHOD FOR NEURAL STEM CELL CULTURE

(71) Applicant: University of Ottawa, Ottawa (CA)

(72) Inventors: Andrew Edward PELLING, Ottawa (CA); Krystal Lee Anne WALKER, Ottawa (CA); Lauren Juile COUVRETTE, Ottawa (CA)

(21) Appl. No.: 19/478,115

(22) PCT Filed: Apr. 26, 2024
(86) PCT No.: PCT/CA2024/050565

§ 371 Date: Oct. 23, 2025

Related U.S. Application Data
(60) Provisional application No. 63/462,408, filed on Apr. 27, 2023.

(51) Int. Cl. C12N 5/0793; C12N 5/079

(52) U.S. Cl. CPC C12N 5/0619; C12N 5/0622; C12N 2500/90; C12N 2501/11; C12N 2501/115; C12N 2506/08; C12N 2513/00; C12N 2533/32; C12N 2533/78; C12N 2537/00; C12N 2539/00

(57) ABSTRACT

A vascular plant based cellulose scaffold for in-vitro production or growth of neural stem cells is provided. The scaffold may be obtained by decellularizing a vascular plant or a part thereof and is capable of supporting the growth of neural stem cells. The scaffold may be coated with any traditional coating known in the art such as a biomolecule, a synthetic biomolecule (Poly-L-Ornithine), a ligand, a protein, an amino acid, an antibody, an extracellular matrix, fibrin, laminin, collagen, fibronectin, poly-l-lysine, a small molecule, a hydrogel, or a matrigel. An in-vitro method of growing or producing neural stem cells is also provided.

### FIELD OF THE INVENTION

The present invention relates to production or growth of neural stem cells for regenerative medicine or stem cell based therapies. More particularly, the invention relates to a vascular plant based scaffold that acts as a substrate for in-vitro production or growth of neural stem cells. The invention also provides methods of in-vitro production or growth of neural stem cells.

### BACKGROUND OF THE INVENTION

Neural stem cells (NSCs) are self-renewing cells that proliferate in vitro and maintain the capacity to differentiate into neurons, astrocytes, and oligodendrocytes. NSC transplantation is being investigated as a therapeutic strategy for numerous disorders of the central nervous system and many preclinical studies report promising results.

NSC based therapies are at the forefront of regenerative medicine strategies for various neural defects and injuries such as stroke, traumatic brain injury and spinal cord injury. However, there are currently important limitations to the efficacy of NSC therapies such as low transplant survival and poor efficiency of neuronal differentiation. To overcome these issues, biocompatible scaffolds have emerged to support cell survival and to direct differentiation and are being used as a vehicle to engraft NSCs while supporting survival of the transplanted cells.

The mammalian spinal cord has limited regenerative capacity after spinal cord injury (SCI). Failure of the adult central nervous system to regenerate can be attributed in part to the limited capacity of injured neurons to re-establish functional axons across the lesion. Mechanical trauma to the tissue elicits a range of cell responses, including increased proliferation of ependymal cells that line the central canal. Upon activation, ependymal cells re-express neural stem cell properties and migrate to the site of injury, where they spontaneously differentiate into oligodendrocytes and astrocytes. Although ependymal cells can differentiate into neurons in vitro, the same has not been demonstrated following SCI in vivo, likely owing to the particularities of the post-SCI environment. Complex pathological mechanisms create a hostile microenvironment at the injury epicenter, which inhibits the regrowth of axons and damages surrounding tissue. For instance, SCI-induced death of oligodendrocytes leads to an accumulation of myelin breakdown products at the injury site. Molecules such as Nogo-A and myelin-associated glycoprotein are released from damaged myelin into the extracellular matrix after SCI and represent an important group of axonal growth inhibitors. Many therapeutic strategies aim to establish a more permissive environment for regeneration by removing inhibitory molecules, providing trophic support, directing stem cell fate, remyelinating axons, or implanting biomaterial scaffolds that can guide axonal growth. In recent years, multimodal therapies have generated much attention since they address multiple aspects of SCI pathology and often produce greater benefit than their individual components. Such therapeutic strategies have combined cell transplants, biomaterials, locomotor training, and neurotrophic factors with the goal of modulating the injury microenvironment to promote repair.

Developing biocompatible scaffolds to bridge the SCI has been at the forefront of tissue engineering strategies. Implantable biomaterials not only provide structural scaffolding to guide cell attachment and migration but can also be used to regulate the inflammatory response or deliver other therapeutics such as stem cells and growth factors. Regenerative biomaterials for SCI are designed to replicate the properties of spinal cord tissue and should therefore have mechanical strength, porosity and internal microstructures that are suitable for neurite extension across the injury site. Moreover, the surface chemistry of biomaterials is of particular importance, as it can be utilized to influence cell migration and differentiation.

Both degradable and non-degradable biomaterials have been investigated for SCI repair. In order for degradable scaffolds to act as extracellular matrix substitutes, it is essential that their rate of degradation aligns with the regeneration rate of axons. Synthetic materials are attractive candidates for spinal cord tissue engineering due to their controllable mechanical properties and high batch-to-batch consistency. For example, polyethylene glycol is a synthetic polymer that can be cross-linked to form injectable hydrogels that provide a framework for regenerating tissue and were shown to promote motor recovery in a rat model of SCI. In addition, poly(lactic-co-glycolic acid)-based scaffolds have produced significant motor improvements and increased tissue remodelling in African green monkeys with an incomplete SCI. However, many synthetic biomaterials are limited by their hydrophobicity, which can impair cell attachment, or by their potential to release toxic by-products during degradation.

### BRIEF DESCRIPTION OF DRAWINGS

FIG. 1 shows cross sections of decellularized Asparagus officinalis scaffolds. FIG. 1A provides an overview of cellulose scaffold (Scale bar=1 mm). FIG. 1B provides SEM of scaffold cross section (Scale bar=200 μm). FIG. 1C provides SEM of vascular bundles within scaffold cross section (Scale bar=100 μm). FIG. 1D provides SEM of scaffold cross section coated with poly-I-ornithine (Scale bar=100 μm).

FIG. 2 provides confocal microscopy (maximum intensity projection) of adult rat NSCs cultured on decellularized asparagus scaffold. F-actin was stained with Fluorescein Phalloidin, Nuclei were stained with Hoechst 33342 (blue) and cellulose was stained with Congo Red. FIG. 2A provides NSCs grown on Poly-I-ornithine (PLO) coated scaffold for 72 hours (Scale bar=500 μm). FIG. 2B provides NSCs grown on uncoated scaffold for 14 days (Scale bar=300 μm). FIG. 2C provides higher magnification sagittal section of NSCs on PLO coated scaffold (Scale bar=100 μm). FIG. 2D provides higher magnification cross section of NSCs on PLO coated scaffold (Scale bar=100 μm) where white arrows indicate neurite-like processes.

FIG. 3 provides percent reduction of AlamarBlue reagent by NSCs grown on a 3D cellulose scaffold (green) compared to 2D control (grey) over 5 days in culture. (Error bars represent standard deviation, N=3 for each condition).

FIG. 4 provides NSC lineage analysis by immunostaining. The figure provides representative confocal microscopy (maximum intensity projections) of adult rat NSCs after 7 days in culture in differentiation media. Nuclei were stained with Hoechst 33342 (blue). FIG. 4A shows NSCs grown on PLO-coated culture plates (2D) stained for GFAP (green). FIG. 4B shows NSCs grown on PLO coated scaffold (3D) stained for GFAP (green). FIG. 4C shows NSCs grown on PLO-coated culture plates (2D) stained for βIII-tubulin (red). FIG. 4D shows NSCs grown on PLO-coated scaffold (3D) stained for βIII-tubulin (red). (Scale bars=50 μm).

FIG. 5 shows in-vitro SEM of 20 μg/ml PLO coated asparagus scaffold.

FIG. 6 shows in-vitro SEM of 40 μg/ml PLO coated asparagus scaffold.

FIG. 7 shows in-vitro SEM of 100 μg/ml PLO coated asparagus scaffold.

FIG. 8 shows 20 μg/ml PLO coated asparagus scaffold seeded with fetal rat neural stem cells.

FIG. 9 shows in vitro cell culture fetal rat neural stem cells confocal microscopy (maximum intensity projection). F-actin was stained with Fluorescein Phalloidin, nuclei were stained with Hoechst 33342 (blue) and cellulose was stained with Congo Red.

FIG. 10 shows in vitro cell culture rat adult hippocampal neural stem cells confocal microscopy (maximum intensity projection). F-actin was stained with Fluorescein Phalloidin, nuclei were stained with Hoechst 33342 (blue) and cellulose was stained with Congo Red.

FIG. 11 provides Fourier transform infrared analysis (FTIR) of PLO coated asparagus scaffold.

FIG. 12 shows in-vivo subcutaneous implant (20 g/ml PLO) H&E stained showing cell infiltration into the scaffolds 1 week, 4 weeks, 8 weeks and 12 weeks.

FIG. 13 shows in vivo subcutaneous implant (20 μg/ml PLO) stained for CD45 showing minimal foreign body response at 12 weeks.

FIG. 14 shows in-vivo subcutaneous implant (20 μg/ml PLO) stained for CD31 demonstrating that the scaffold becomes vascularized at 1 week, 4 weeks, 8 weeks and 12 weeks.

FIG. 15 shows visible attachment of NSC's to matrigel, which is clearly evident but is not optimal. FIG. 15A shows attachment to Matrigel 1:25, FIG. 15B shows attachment to Uncoated Scaffold and FIG. 15C shows attachment to PLO coated Scaffold.

FIG. 16 shows plant-derived cellulose biomaterial in rodent model of complete transection spinal cord injury. FIG. 16A shows decellularized plant-derived scaffold consisting of vascular bundles and parenchyma (scale bar=1 mm). FIG. 16B shows SEM of cellulose scaffold microarchitecture (scale bar=100 μm). FIG. 16C shows hematoxylin & eosin staining of cross section of cellulose scaffold after 12 weeks in vivo. The scaffold's vascular bundles are infiltrated with host cells. (Scale bar=200 μm). FIG. 16D shows an exposed fully transected spinal cord (left box) and a scaffold after implantation (right box). FIG. 16E shows hematoxylin & eosin staining of sagittal section of spinal cord tissue with cellulose scaffold (outlined) after 12 weeks in vivo (Scale bar=2 mm). FIG. 16F shows immunohistochemistry staining for CD31 (brown) in a sagittal section at the biomaterial-tissue interface (representative image from the ASP group). CD31 positive blood vessels are denoted by arrow (Scale bar=200 μm). FIG. 16G shows immunohistochemistry staining for CD31 (brown) in a sagittal section at the biomaterial-tissue interface (representative image from the PLO group). CD31 positive blood vessels are denoted by arrow (Scale bar=200 μm).

FIG. 17 shows locomotor assessments after complete spinal cord transection. FIG. 17A shows average BBB score of each experimental group at week 2 after SCI compared to week 11 after SCI (2way ANOVA Uncorrected Fisher's LSD; ****P<0.0001, *P=0.0158; mean±s.e. m, n=11 PLO animals, n=9 ASP animals, n=8 No Tx animals). FIG. 17B shows average KSAT swim assessment score of each experimental group at week 2 after SCI compared to week 11 after SCI (2way ANOVA Uncorrected Fisher's LSD; ***P=0.0001; mean±s.e.m, n=11 PLO animals, n=9 ASP animals, n=8 No Tx animals). FIG. 17C shows average maximum angle achieved by each experimental group in inclined plane assessment at final week (mean±s.e.m, n=11 PLO animals, n=9 ASP animals, n=8 No Tx animals).

FIG. 18 shows 5-HT immunohistochemistry staining of sagittal section of T8-T9 spinal cord injury. FIG. 18A shows 5-HT staining of spinal cord implanted with ASP scaffold (as outlined). IHC was performed using DAB as the chromogen (brown) (Scale bar=2 mm). FIG. 17B shows 5-HT staining of spinal cord with no implant. IHC was performed using DAB as the chromogen (brown) (Scale bar=2 mm).

FIG. 19 shows retrograde tract tracing of ascending sensory afferents by CTb injection into sciatic nerve. FIG. 19A shows furthest rostral CTb-traced axons. Each point represents the distance (mm) between the injury epicenter and the furthest rostral CTB-traced axon in one animal. Bar represents the mean±s.e.m of each group (One-way ANOVA; *P=0.0264, n=4 PLO animals, n=2 ASP animals, n=3 No Tx animals). FIG. 19B shows hoescht (blue) stained cross section of spinal cord at T10 (caudal to the injury) confirms presence of CTb (red) in dorsal column and lamina 4 (Scale bar 300 μm). FIG. 19C shows Hoescht (blue) stained sagittal section of spinal cord from SCI animal with no scaffold. CTb-traced axons (red) can be seen in the dorsal column (Scale bar 500 μm). FIG. 19D shows Hoescht (blue) stained sagittal section of spinal cord from SCI animal with uncoated scaffold. CTb-traced axons (red) can be seen in the dorsal column (Scale bar 500 μm). FIG. 19E shows Hoescht (blue) stained sagittal section of spinal cord from SCI animal implanted with PLO-coated cellulose scaffold. CTb-traced axons (red) can be seen in the dorsal column (Scale bar 500 μm).

FIG. 20 shows anterograde labeling of the corticospinal tract by injection of dextran amine into hindlimb motor cortex. FIG. 20A shows furthest caudal dextran amine-traced axons. Each point represents the distance (mm) between the injury epicenter and the furthest caudal DA-traced axon in one animal. Bar represents the mean±s.e. m of each group (One-way ANOVA; P=0.0903, n=4 PLO animals, n=4 ASP animals, n=3 No Tx animals). FIG. 20B shows hoescht (blue) stained cross section of spinal cord at T6 (rostral to the injury) confirms presence of dextran amine (green) in corticospinal tract (Scale bar 300 μm). FIG. 20C shows hoescht (blue) stained sagittal section of spinal cord from SCI animal with no scaffold. DA-traced axons (green) can be seen in the corticospinal tract (Scale bar 500 μm). FIG. 20D shows hoescht (blue) stained sagittal section of spinal cord from SCI animal implanted with uncoated cellulose scaffold. DA-traced axons (green) can be seen in the corticospinal tract (Scale bar 500 μm). FIG. 20E shows hoescht (blue) stained sagittal section of spinal cord from SCI animal implanted with PLO-coated cellulose scaffold. DA-traced axons (green) can be seen in the corticospinal tract (Scale bar 500 μm).

FIG. 21 shows immunostaining for β-III tubulin and neurofilament-200 reveals neural cells attaching to scaffold and migrating along channels. FIG. 21A shows β-III tubulin (red) and Hoescht (blue) staining of sagittal section within PLO-coated biomaterial. Cell bodies (identified by arrow) can be seen inside the scaffold (scale bar 50 μm). FIG. 21B β-III tubulin (red) and Hoescht (blue) staining of sagittal section within PLO-coated biomaterial. Axons (identified by arrow) can be seen sprouting inside the scaffold (scale bar 50 μm). FIG. 21C shows neurofilament 200 (green) and Hoescht (blue) staining of sagittal section at the interface (dashed line) between PLO-coated biomaterial and spinal cord (scale bar 200 μm). FIG. 21D shows Neurofilament 200 (green) and Hoescht (blue) staining of sagittal section at the interface (dashed line) between PLO-coated biomaterial and spinal cord. NF200 positive cells can be seen infiltrating the biomaterial (asterisk) & NF200 axon projections extend from the dorsal to ventral aspect of the biomaterial (arrow) (scale bar 50 μm).

FIG. 22 shows luxol fast blue (LFB) staining of sagittal tissue sections for assessment of myelin at the site of spinal cord injury. FIG. 22A shows representative LFB staining at scaffold-tissue interface in animals implanted with PLO coated cellulose scaffold. Outline denotes cellulose scaffold (Scale bar=200 μm). FIG. 22B shows LFB staining at scaffold-tissue interface in animals implanted with uncoated cellulose scaffold (outlined) (Scale bar=200 μμm). FIG. 22C shows LFB staining at cyst-tissue interface in animals with no scaffold (Scale bar=200 μm).

FIG. 23 shows luxol fast blue (LFB) staining of sagittal tissue sections for assessment of myelin at the site of spinal cord injury. FIG. 23A shows representative LFB staining at scaffold-tissue interface in animals implanted with PLO coated cellulose scaffold. Outline denotes cellulose scaffold (Scale bar=200 μm). FIG. 23B shows LFB staining at scaffold-tissue interface in animals implanted with uncoated cellulose scaffold (outlined) (Scale bar=200 μm). FIG. 23C shows LFB staining at cyst-tissue interface in animals with no scaffold (Scale bar=200 μm).

FIG. 24 shows immunostaining for glial fibrillary acidic protein (GFAP) in sagittal spinal cord sections after SCI. FIG. 24A shows GFAP (green) stained cord section from animal implanted with PLO coated cellulose scaffold. Nuclei stained with DAPI (blue). Outline denotes cellulose scaffold (Scale bar=200 μm). FIG. 24B shows GFAP (green) stained cord section from animal implanted with uncoated cellulose scaffold (outlined). Nuclei stained with DAPI (blue). (Scale bar=200 μm). FIG. 24C shows GFAP (green) stained cord section from animal with no scaffold. Nuclei stained with dapi (blue) (Scale bar=200 μm).

### SUMMARY OF THE INVENTION

The present invention teaches a vascular plant based cellulose scaffold for in-vitro production or growth of neural stem cells. In some embodiments, the plant-based scaffold is suitable for in-vitro culture or in-vitro growth of neural stem cells. The scaffold may be obtained by decellularizing the vascular plant or a part thereof. In some embodiment, the scaffold may optionally be obtained by mercerizing the vascular plant of part thereof. In some embodiments, the scaffold is capable of supporting the growth of the neural stem cells.

The scaffold may be coated with one or more coatings known in the art. For instance, the scaffold may be coated with a biomolecule, a synthetic biomolecule, a ligand, a protein, an amino acid, an antibody, an extracellular matrix, fibrin, laminin, collagen, fibronectin, poly-l-lysine, a small molecule, a hydrogel, or a matrigel. In certain embodiments, the scaffold is coated with a synthetic biomolecule. such as Poly-L-Ornithine (PLO).

The vascular plant based scaffold as defined above may be used or employed in regenerative medicine or for production or growth of biopharmaceuticals. The scaffold may also be used or employed in neural tissue engineering, or in the treatment of neural disorders, defects or injuries. The vascular plant based scaffold may be used or employed in in-vitro drug production, growth or in-vitro drug testing. The scaffold may also be used or employed in in-vitro production or growth of neural cell proteins or in-vitro harvesting of neural cell proteins. The scaffold may also be employed for production or growth of neural transplant or neural cell banks or for use in production or growth of a neural construct for in-vitro disease modeling.

A method of in-vitro production or growth of neural stem cells is provided. The method involves seeding a culture medium comprising the neural stem cells in a container on a vascular plant based cellulose scaffold as defined above and allowing the neural stem cells to attach and grow on the cellulose scaffold for a predetermined amount of time. The pre-determined amount of time may range from at least 24 hours to 5 days.

The method may involve a step of coating the container with a natural biomolecule, a synthetic biomolecule, a ligand, a protein, an amino acid, an antibody, an extracellular matrix, fibrin, laminin, collagen, fibronectin, poly-l-lysine, a small molecule, a hydrogel, or a matrigel, prior to the step of seeding the vascular scaffold. For instance, the method may involve coating the container with poly-L-ornithine and laminin. The method may involve a pre-processing step prior to the step of seeding the scaffold. The pre-processing step may be a step of sterilizing the scaffold with 70% ethanol, washing the scaffold with a saline solution, a detergent solution, a physiological buffer or a salt solution, or pre-treating the surface of the scaffold. The washing step may additionally involve an incubation step in which the scaffold is incubated in the salt solution overnight.

The surface of the scaffold may be pre-treated by treating the surface chemically, treating the surface physically, functionalizing the surface, succinylating the surface or a combination thereof. For instance, the surface may be pre-treated by treating the surface with a biomolecule, a synthetic biomolecule, a ligand, a protein, an amino acid, an antibody, an extracellular matrix, fibrin, laminin, collagen, fibronectin, poly-l-lysine, a small molecule, a hydrogel, a matrigel or a pharmaceutically acceptable compound. The surface may be chemically treated with a synthetic biomolecule such as poly-L-ornithine.

### DETAILED DESCRIPTION

The following description is of preferred embodiments by way of example only, and without limitation to the combination of features necessary for carrying the invention into effect.

All terms are intended to be understood as they would be understood by a person skilled in the art. Unless defined otherwise, all technical and scientific terms used herein have the same meaning as commonly understood by one of ordinary skill in the art to which the disclosure pertains. The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application. Accordingly, the terminology used herein is for the purpose of describing particular embodiments only, and is not intended to be limiting.

### DEFINITIONS

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof and their grammatical equivalents as used herein, can be used interchangeably. These terms can convey that any and all combinations are specifically contemplated. The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” “alternate embodiment”, or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value. The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

Cell scaffolds can enhance therapeutic efficacy of Neural Stem Cells (NSCs) by promoting strong cell adhesion, guiding cell migration, and shielding transplanted NSCs from the cytotoxic injury environment occurring in Central Nervous System (CNS) injuries.

Since scaffolds provide three-dimensional physical support for cell-growth they are considered a 3D cell culture system. This system more accurately represents the in vivo microenvironment compared to traditional 2D culture and the behavior of cells within 3D systems is more physiologically relevant, making it a better cell culturing method for tissue engineering and drug discovery. As such, various scaffold-based neural constructs have been developed for use in disease modeling, regenerative medicine, and the study of the stem cell niche.

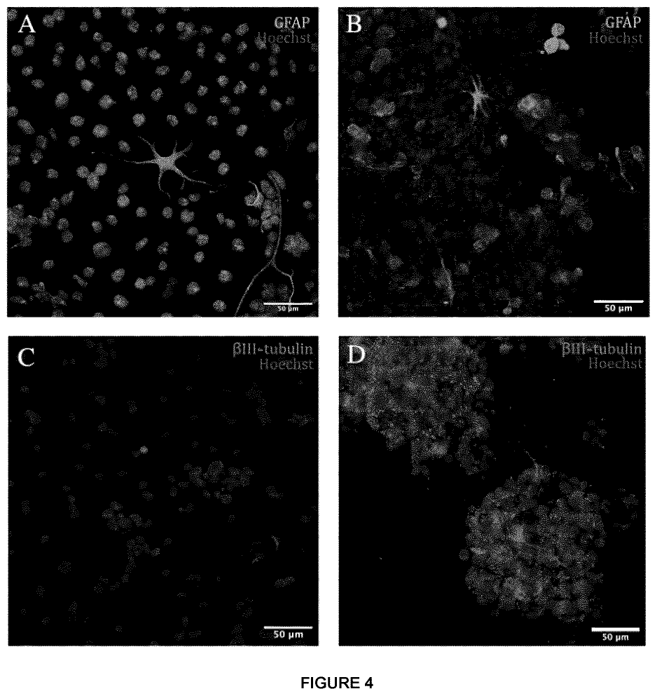

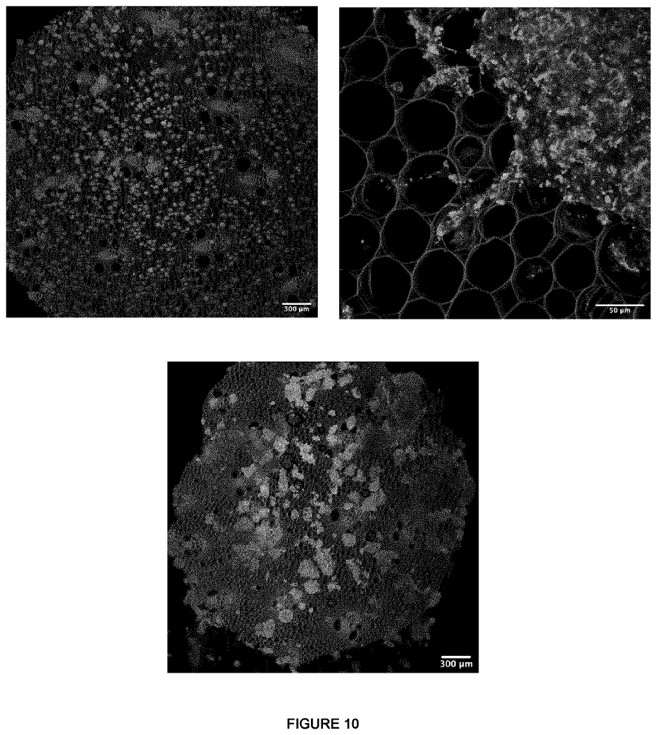

In the present invention, the inventors investigated decellularized plant tissue as a novel scaffold for three-dimensional in vitro culture or in-vitro production or growth of NSCs. The proposed plant cellulose scaffolds were shown to support attachment and proliferation of adult rat hippocampal neural stem cells (NSCs) as shown in the appended figures (FIG. 10). Further, NSCs differentiated on the cellulose scaffold had significant increases in their expression of neuron-specific beta-III tubulin and glial Fibrillary Acidic Protein compared to 2D culture, indicating that the scaffold may enhance differentiation of NSCs towards astrocytic and neuronal lineages (FIG. 4). These findings suggest that plant-derived cellulose scaffolds have the potential to be used in neural tissue engineering and that their native surface topography can be harnessed to direct differentiation of NSCs.

The inventors investigated the possibility of producing plant-derived cellulose biomaterials, which were shown to support cell infiltration and vascularization in vivo. In recent years, various plant tissues have been decellularized to produce biocompatible scaffolds for culturing mammalian cells, for pre-clinical applications including skin tissue, neural tissue, and bone tissue engineering. For example, Dickie et al. decellularized spinach leaves while maintaining their vascular architecture, which was then repopulated with human dermal microvascular endothelial cells. Furthermore, Flammulina velutipes mushroom has been successfully used as a nerve guidance conduit in a rat model of sciatic nerve defection.

As evidenced above, plant-derived biomaterials are becoming increasingly attractive for biomedical applications, which can be attributed in part to improved cost effectiveness, scalability, and lower immunogenicity relative to animal sources.

In the present invention, the viability of a plant-derived biomaterial as a 3D in vitro culture system for adult rat neural stem cells was investigated. In an exemplary embodiment, the asparagus scaffold's physical characteristics and mechanical testing was investigated by scanning electron microscopy. A person skilled in the art would readily understand and appreciate that any vascular plant-material or part thereof can be used to prepare the proposed scaffold.

Additionally, the scaffold's ability to support attachment and migration of NSCs was assessed for various time periods. The differentiation potential of neural stem cells in this 3D culture system by immunostaining for markers including neuron-specific β-III Tubulin and glial fibrillary acidic protein was also investigated.

### EMBODIMENTS

In an embodiment of the invention, a vascular plant based cellulose scaffold for in-vitro production or growth of neural stem cells is provided. In some embodiments, the plant-based scaffold is suitable for in-vitro culture or in-vitro growth of neural stem cells. The scaffold may be obtained by decellularizing the vascular plant or a part thereof. In some embodiment, the scaffold may optionally be obtained by mercerizing the vascular plant of part thereof. In some embodiments, the scaffold is capable of supporting the growth of the neural stem cells.

In the present invention, any vascular plant known in the art can be employed to derive the plant-based scaffold. In some embodiments, the vascular plant may be asparagus, celery, ferns, horsetails, conifers, flowering plants, or clubmosses.

In some embodiments, the scaffold may be coated with one or more coatings known in the art. In certain embodiments, the scaffold may be coated with a biomolecule, a synthetic biomolecule, a ligand, a protein, an amino acid, an antibody, an extracellular matrix, fibrin, laminin, collagen, fibronectin, poly-l-lysine, a small molecule, a hydrogel, or a matrigel. In certain embodiments, the scaffold is coated with a synthetic biomolecule. In certain embodiments, the scaffold is coated with Poly-L-Ornithine (PLO).

In some embodiments, the scaffold may have a porous structure comprising a plurality of vascular bundles with varying diameters and a plurality of parenchymal cells. In certain embodiments, the vascular bundles may be interspersed between the parenchymal cells. In some embodiments, the vascular bundles may be unevenly spaced apart from each other and traverse the length of the scaffold. The parenchymal cells form the surface of the scaffold.

The porous structure of the scaffold is capable of promoting attachment, proliferation or differentiation of the neural stem cells. In certain embodiments, the porous structure of the scaffold may allow the neural stem cells to maintain its differentiation ability. In some other embodiments, the porous structure of the scaffold is capable of promoting differentiation of the neural stem cells into neurons, dopaminergic neurons, motor neurons, astrocytes, oligodendrocytes or a combination thereof. In some embodiments, the porous structure of the scaffold promotes or supports differentiation of the neural stem cells into neurons and astrocytes.

In the embodiments, where the scaffold is coated with poly-L-ornithine, the coating increases production or growth of the neural stem cells by promoting filopodia formation. In some embodiments, the porous structure of the scaffold promotes or supports increased production or growth of the neural stem cells by enabling nutrient exchange, oxygen exchange and removal of waste within the scaffold.

The porosity of the scaffold may range from 10% to 95% and the scaffold may have an elastic modulus in the range of 1 kPa to 1000 kPa. In certain embodiments, the scaffold is biocompatible, biodegradable or both.

In certain embodiments, the surface of the scaffold may be modified by a chemical treatment or a physical treatment. The modification may enhance attachment of the neural stem cells to the surface of the scaffold. In certain embodiments, the surface of the scaffold may be treated by functionalizing the surface, gamma irradiation, radical treatment, oxidative treatment, or a combination thereof.

In the above embodiments, the functionalization may be achieved by providing a functional group that creates a charge on the surface of the scaffold. This may be achieved by mixing the scaffold with the functional group, or allowing the functional group to interact with the scaffold for a pre-determined amount of time, or any other procedure known in the art. The functional group may be a primary amine, a tertiary amine, a quaternary compound, an alcoholic group, a carboxylic acid group, an aldehyde group, a sulfonyl group or a combination thereof. In certain embodiment, the functional group may be an alkene, an alkyne, an amine, a ketone, an amide, an ester, a nitrile group or an ether.

In some embodiments, the scaffold promotes or supports three-dimensional growth of the neural stem cells where the neural stem cells form neurospheres on the scaffold. In certain embodiments, the scaffold may promote or support the cells to grow neurite-like processes. In certain other embodiments, the scaffold may promote or support growth of multiple layers of the neural stem cells. In some further embodiments, the scaffold may promote or support production or growth of neural cell proteins.

In some embodiments, a vascular plant based scaffold as defined above may be used or employed in regenerative medicine or for production or growth of biopharmaceuticals. In certain embodiments, the scaffold may be used or employed in neural tissue engineering, or in the treatment of neural disorders, defects or injuries. In certain specific embodiments, the vascular plant based scaffold may be used or employed in in-vitro drug production, growth or in-vitro drug testing. In some other embodiments, the scaffold may be used or employed in in-vitro production or growth of neural cell proteins or in-vitro harvesting of neural cell proteins. The scaffold may also be employed for production or growth of neural transplant or neural cell banks or for use in production or growth of a neural construct for in-vitro disease modeling.

In certain embodiments, a method of in-vitro production or growth of neural stem cells is provided. The method involves seeding a culture medium comprising the neural stem cells in a container on a vascular plant based cellulose scaffold as defined above and allowing the neural stem cells to attach and grow on the cellulose scaffold for a predetermined amount of time. In certain embodiments, the pre-determined amount of time ranges from at least 24 hours to 5 days. In certain specific embodiments, the pre-determined amount of time is at least 48 hours.

In certain embodiments, the culture medium is infused with growth promoting nutrients or growth factors such as enzymes, antigens, specialized proteins, inhibitors, stem cell factors, binders, stimulating factors, that promote, facilitate or increase attachment and growth of the neural stem cells to the scaffold.

In certain embodiments, the culture medium is selected from any cell-culture medium known in the art. In some embodiments, the culture medium is selected from any medium known for promoting the growth of neural stem cells. In some embodiment, the culture medium is DMEM MEM/F12, Aplha MEM, Knockout DMEM/F12, FBS, FCS, Goat Serum, or Horse Serum. In some other embodiments, the culture medium is A: 1X KnockOut DMEM/F-12 supplemented with 2% B 27 (cat. 17504044 ThermoFisher), 2 mM GlutaMAX-I (ThermoFisher), B: serum free medium (1X KnockOut D-MEM/F-12 with 2% StemPro Neural Supplement (ThermoFisher A1050801), 20 ng/ml bFGF, 20 ng/ml EGF, or 2 mM GlutaMAX-I.

In certain embodiments, the method may be carried out at a temperature ranging from 30 °C to 45 °C. In some embodiments, the method is carried out within a temperature range of 32 °C to 42 °C In some embodiments, the method may be carried out at a pH range known to support the growth of neural stem cells in the art. In certain embodiments, the method is carried out at a pH ranging from 6.8 to 7.2.

In some embodiments, the method may be carried out in any growth chamber known in the art. In certain embodiments, the method may be carried out in a container such as a flask, a culture vessel, a bioreactor, a petri dish, a multi-well plate or a glass chamber.

In certain embodiments, the method may involve a step of coating the container with a natural biomolecule, a synthetic biomolecule, a ligand, a protein, an amino acid, an antibody, an extracellular matrix, fibrin, laminin, collagen, fibronectin, poly-l-lysine, a small molecule, a hydrogel, or a matrigel, prior to the step of seeding the vascular scaffold. In some embodiments, the container is coated with a natural biomolecule or a synthetic biomolecule or both. In some embodiments, the method involves coating the container is coated with poly-L-ornithine and laminin. The poly-L-ornithine coating is capable of increasing production or growth of the neural stem cells by promoting filopodia formation.

In some embodiments, the method involves a pre-processing step prior to the step of seeding the scaffold. The pre-processing steps may include a step of sterilizing the scaffold with 70% ethanol, washing the scaffold with a saline solution, a detergent solution, a physiological buffer or a salt solution, or pre-treating the surface of the scaffold. In certain embodiments, the washing step may additionally involve an incubation step in which the scaffold is incubated in the salt solution overnight.

In certain embodiments, the surface of the scaffold may be pre-treated by treating the surface chemically, treating the surface physically, functionalizing the surface, succinylating the surface or a combination thereof. In some embodiments, the surface may be pre-treated by treating the surface with a biomolecule, a synthetic biomolecule, a ligand, a protein, an amino acid, an antibody, an extracellular matrix, fibrin, laminin, collagen, fibronectin, poly-l-lysine, a small molecule, a hydrogel, a matrigel or a pharmaceutically acceptable compound. In one embodiment, the surface is chemically treated with a synthetic biomolecule for chemical treatment using poly-L-ornithine. The pre-treatment allows modification of the surface of the scaffold and enhances production or growth of cells by increasing attachment to the surface of the scaffold.

In some embodiments, the surface of the scaffold is functionalized by providing a functional group that creates a charge on the surface of the scaffold. The functional group may be selected from any functional group know in the art. The functional group may be a primary amine, a tertiary amine, a quaternary compound, an alcoholic group, carboxylic acid, aldehyde, sulfonyl or a combination thereof. In some embodiments, the functional group may be an alkene, an alkyne, an amine, a ketone, an amide, an ester, a nitrile group and an ether.

In some embodiments, the method involves a sterilization step prior to step of seeding the scaffold, wherein the scaffold is sterilized for a pre-determined period of time. In some embodiments, the sterilization step may be carried out by autoclaving, treatment with 70% ethanol, gamma irradiation, or treatment with ethylene oxide. The sterilization step may be carried out for 20 minutes to several days.

In some embodiments, the production or growth of neural stem cells by the above method can be increased by modulating stiffness of the scaffold. In some embodiments, the production or growth of neural stem cells may be increased by modulating anisotropy of the scaffold.

In certain embodiments, the method further comprises a quantification step to quantify the production or growth of the neural stem cells. The quantification step may be carried out by any cell quantification assay known in the art. In certain embodiments, the quantification may be carried out by a biomolecule quantification assay, cell quantification assay, cell viability assay, image analysis, immunohistochemistry or electrophysiology. In some embodiments, the quantification step may be followed by an optimization step to optimize the growth conditions in the container, to optimize the quantity of scaffold, to optimize the size of the scaffold, to optimize the structure of the scaffold or a combination thereof.

In some embodiments, the method further comprises an incubation step wherein the scaffold and the neural stem cells are incubated at a pre-determined temperature. In certain embodiments, the incubation step may be carried out at a temperature ranging from 25 °C-50 °C, preferably within 30 °C-45 °C

In some embodiments, the scaffold and the neural stem cells may be incubated with a pre-determined concentration of CO2, at a pre-determined temperature and for a pre-determined amount of time. In some specific embodiments, the scaffold and the neural stem cells may be incubated at 37 °C with 5% CO₂.

Attachment, proliferation, growth and differentiation of neural stem cells on the scaffold can be achieved using the above-recited method and various embodiments. The neural stem cells produced are capable of differentiating to neurons, astrocytes, oligodendrocytes, or a combination thereof after attaching to the scaffold. In certain embodiments, the neural stem cells differentiate to neurons and astrocytes after attaching to the scaffold. The neural stem cell differentiation may be determined by any known assay in the art. For instance, the differentiation can be determined using an immunostaining assay, cell imaging, confocal microscopy, flow cytometry, or electrophysiology.

### Experimental Discussion

Spinal cord injury (SCI) is a debilitating neurological condition with far-reaching consequences for patients, including loss of motor function and significant limitations to quality of life. Implantable biomaterials have emerged as a therapeutic strategy to modulate the SCI microenvironment and facilitate regeneration of axons.

Recently, plant-derived scaffolds have been investigated in vivo for various biomedical applications including skin, tendon, nerve and bone tissue engineering. Natural materials such as collagen, fibrin, alginate, chitosan and hyaluronic acid are widely studied as regenerative scaffolds for SCI due to their excellent biocompatibility and cell adhesion properties. In particular, collagen-based scaffolds are a promising SCI treatment that has been explored in diverse forms including collagen hydrogels, collagen sponges or collagen scaffolds that deliver therapeutics to the injury. Ongoing human clinical trials have reaffirmed the therapeutic potential of collagen scaffolds, notably in their ability to promote tissue regeneration and functional recovery. One common issue with scaffolds made from natural polymers is insufficient mechanical strength and durability in vivo, which multiple studies have aimed to resolve by adding cross-linkers or combining natural and synthetic biomaterials.

To overcome these issues, many different plant species can be decellularized with the help of detergents, sonication, enzymatic digestion, or freeze-thawing methods to produce cellulose-based scaffolds composed of β(1→4) linked D-glucose units. Plant cellulose scaffolds have shown to be biocompatible in vivo and supported extracellular matrix deposition when implanted subcutaneously. In addition, these scaffolds became vascularized as early as 1 week post-implantation. Since cellulose is not biodegradable in humans, scaffolds made from this material are poised to offer long-term durability in vivo. Moreover, plant cellulose has proven to be a versatile material that can be combined with hydrogels to generate customizable macroscopic structures. For instance, cellulose scaffolds can be functionalized with chemical coatings to promote cell adhesion.

In the present invention, the inventor propose using plant-derived cellulose scaffolds to support locomotor recovery and neural tissue repair in a rat model of spinal cord injury. In some cases, the plant-derived cellulose scaffolds could be coated with various coatings. In a specific case, the scaffolds can be coated with coated with poly-L-ornithine, a positively charged amino acid chain that is known to promote neural stem cell differentiation into neurons and enhance myelin regeneration.

In an exemplary experiment, upon complete transection of the spinal cord, animals were implanted with a plant-derived scaffold coated in poly-L-ornithine (PLO). Then, recovery of motor function was evaluated by the Basso, Beattie and Bresnahan (BBB) locomotor scale as well as the Karolinska Institutet Swim Assessment Tool (KSAT). Retrograde tracing of ascending sensory tracts revealed enhanced regeneration in animals that received the PLO-coated scaffold. Immunostaining for β-III tubulin and NF200 showed axonal sprouting within the cellulose biomaterial and LFB staining highlights myelination around the PLO-coated scaffold.

For instance, poly-L-ornithine-coated cellulose scaffolds produced from stalks of Asparagus officinalis can support attachment and proliferation of neural stem cells in vitro. In culture, rat neural stem cells form neurospheres that attach to the cellulose biomaterial and readily infiltrate its channels. Increased expression of neuron-specific beta-III tubulin and glial fibrillary acidic protein in cells grown on these scaffolds suggests they promote differentiation of NSCs towards astrocytes and neurons in vitro.

The ability of coated cellulose biomaterials e.g. with poly-L-ornithine, to support neural tissue repair and functional recovery after traumatic SCI. To demonstrate the present invention, the inventors implanted coated scaffolds e.g. with poly-L-ornithine, as well as uncoated scaffolds acutely following a complete spinal cord transection in rats housed in an enriched environment. Motor recovery was monitored by the BBB open field assessment and KSAT. Sprouting and regeneration of the dorsal columns were analyzed by neural tract tracing with the retrograde tracer CTb injected into the sciatic nerve. These results point to enhanced regeneration of the sensory tracts in animals that received coated implants compared to controls. In addition, the descending fibers of the corticospinal tracts were visualized with dextran amine injections in the motor cortex, which revealed CST axons projecting along the cellulose biomaterial. Finally, spinal cord tissue was immunostained for various markers to identify key cell types interacting with the biomaterial. In sum, the inventors demonstrate the ability of surface modified plant-derived scaffolds to promote axonal sprouting in the lesion and induce motor recovery after SCI.

### 1.1. Characterization of Plant Cellulose Scaffold

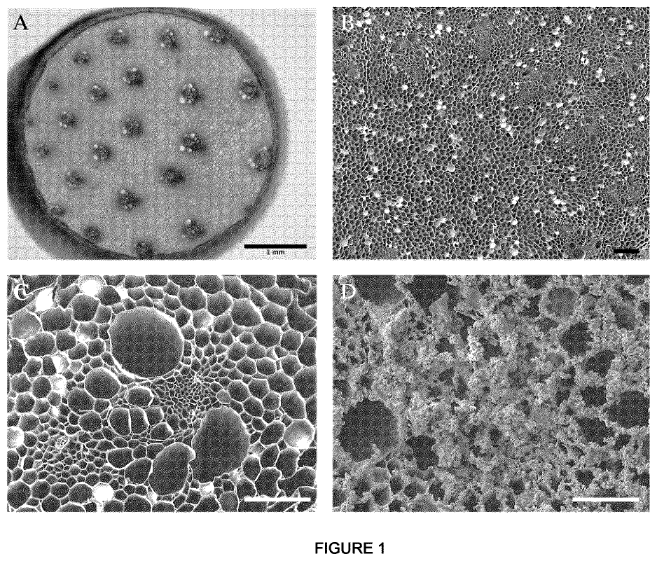

In FIG. 1 cross sections of decellularized Asparagus officinalis scaffolds is shown. FIG. 1A provides an overview of cellulose scaffold (Scale bar=1 mm). FIG. 1B provides SEM of scaffold cross section (Scale bar=200 μm). FIG. 1C provides SEM of vascular bundles within scaffold cross section (Scale bar=100 μm). FIG. 1D provides SEM of scaffold cross section coated with poly-I-ornithine (Scale bar=100 μm).

Raw Asparagus officinalis stalks were cut into discs of 4 mm in diameter and 1.2 mm in height, which were then decellularized to remove all native cells. The resulting scaffold was composed of vascular bundles (VBs) interspersed between parenchymal cells (as shown in FIG. 1A). Naturally occurring structures within the scaffold were characterized by scanning electron microscopy (as shown in FIG. 1B-D). A majority of the scaffold's surface consisted of parenchyma that had an average diameter of 39±15 μm. In addition, each scaffold was found to contain 14±2 vascular bundles, which were aligned channels that traversed the length of the scaffold, with an average spacing of 602±61 μm.

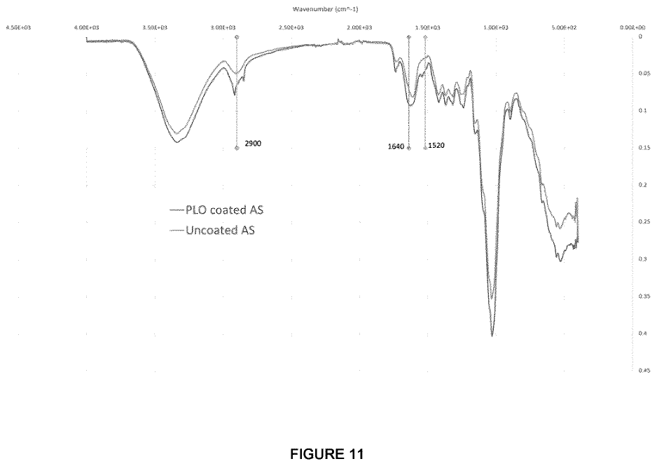

The distribution of channel diameters observed in these scaffolds may be conducive to cell survival since such porous networks have previously been demonstrated to enable nutrient exchange and waste removal within scaffolds. The Young's Modulus of the cellulose scaffold was found to be 430±139 kPa (n=12). Moreover, the scaffolds were coated overnight in Poly-I-ornithine and attachment was confirmed by SEM (FIG. 1D) and Fourier transform infrared (FTIR). FIG. 11 provides the FTIR of PLO coated asparagus scaffold.

1.2. Cellulose scaffold supports rat NSC attachment and proliferation

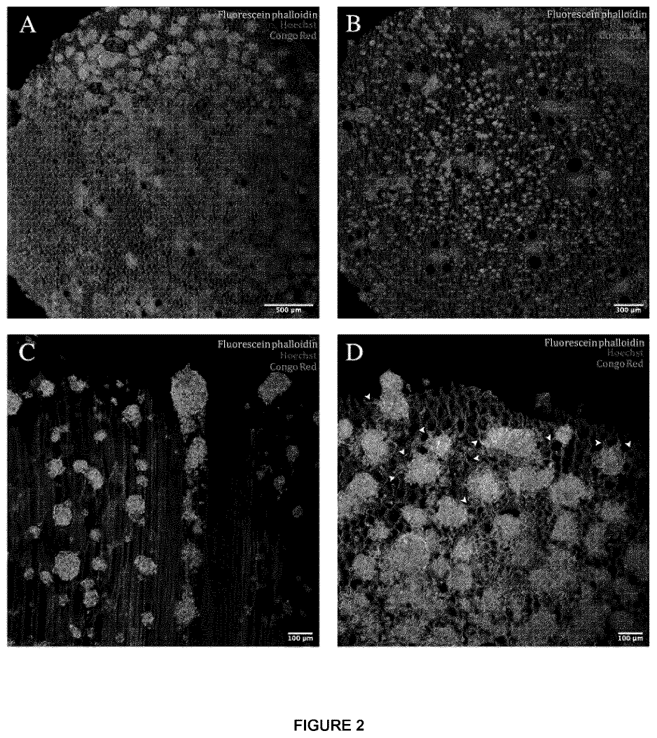

In FIG. 2, confocal microscopy (maximum intensity projection) of adult rat NSCs cultured on decellularized asparagus scaffold is shown. F-actin was stained with Fluorescein Phalloidin, Nuclei were stained with Hoechst 33342 (blue) and cellulose was stained with Congo Red.

FIG. 2A provides NSCs grown on Poly-I-ornithine (PLO) coated scaffold for 72 hours (Scale bar=500 μm). FIG. 2B provides NSCs grown on uncoated scaffold for 14 days (Scale bar=300 μm). FIG. 2C provides higher magnification sagittal section of NSCs on PLO coated scaffold (Scale bar=100 μm). FIG. 2D provides higher magnification cross section of NSCs on PLO coated scaffold (Scale bar=100 μm) where white arrows indicate neurite-like processes.

A single-cell suspension of NSCs was seeded onto scaffolds and, after 72 hours, neurospheres had attached to the cellulose (FIG. 2). Cell attachment was examined by F-actin staining, which revealed substantial cell-ECM interactions and cell-cell interactions. Many neurospheres with diameters of up to 300 μm were found to adhere to the uncoated scaffold (FIG. 2B) and neurite-like processes were observed projecting out from these neurospheres (FIG. 2D). With the addition of a poly-I-ornithine coating, cells migrated out from the attached neurospheres to form a pseudo-monolayer on the scaffold surface (FIG. 2A). Further, many neurospheres as well as individual cells were observed to migrate along the channels of the scaffolds (FIG. 2C).

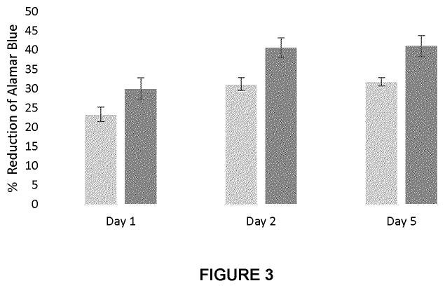

In addition, cell proliferation on the scaffolds was quantified by an AlamarBlue assay, which monitored the chemical reduction of culture media to detect metabolic activity of cells. In FIG. 3 percent reduction of AlamarBlue reagent by NSCs grown on a 3D cellulose scaffold (green) compared to 2D control (grey) over 5 days in culture is shown. (Error bars represent standard deviation, N=3 for each condition).

Over 5 days in culture (FIG. 3), the percentage of reduced AlamarBlue reagent increased progressively, indicating continued growth of NSCs on the scaffold. When compared to NSCs grown as a monolayer (2D control), cells on the scaffold had a slight reduction in metabolic activity during the first 5 days of growth.

### 1.3. Plant Cellulose Scaffold Enhances Neuronal and Astrocytic Differentiation

To determine the effects of this 3D culture system on NSC differentiation potency, the inventors monitored NSCs' expression of lineage specific markers. A single-cell suspension of NSCs was simultaneously seeded onto PLO-coated scaffolds and onto a PLO-coated culture plate at equal seeding densities. After seven days in differentiation media, cells were fixed and immunostained for GFAP (an astrocytic marker) or neuron specific βIII-tubulin (immature neuron marker).

In FIG. 4 NSC lineage analysis by immunostaining is shown. Representative confocal microscopy (maximum intensity projections) of adult rat NSCs after 7 days in culture in differentiation media is provided. Nuclei were stained with Hoechst 33342 (blue).

FIG. 4A shows NSCs grown on PLO-coated culture plates (2D) stained for GFAP (green). FIG. 4B shows NSCs grown on PLO coated scaffold (3D) stained for GFAP (green). FIG. 4C shows NSCs grown on PLO-coated culture plates (2D) stained for βIII-tubulin (red). FIG. 4D shows NSCs grown on PLO-coated scaffold (3D) stained for βIII-tubulin (red). (Scale bars=50 μm).

Interestingly, a significantly higher fraction of GFAP+ cells were observed on the cellulose scaffold (as shown in FIG. 4B) compared to the 2D monolayer culture (as shown in FIG. 4A) (P<0.0001, n=5). Similarly, immunostaining revealed enhanced expression of βII-tubulin on the 3D scaffold. As shown in FIG. 4C, cells in 2D condition were 0.78±0.7% βIII-tubulin+ whereas cells grown on the 3D scaffold were 16.4±4.5% βIII-tubulin+as shown in FIG. 4D, which represents a significant increase in expression of this early neuronal marker (P<0.0001, n=4). Both findings demonstrate that NSCs retained their ability to differentiate into various lineages when cultured in this 3D cellulose scaffold.

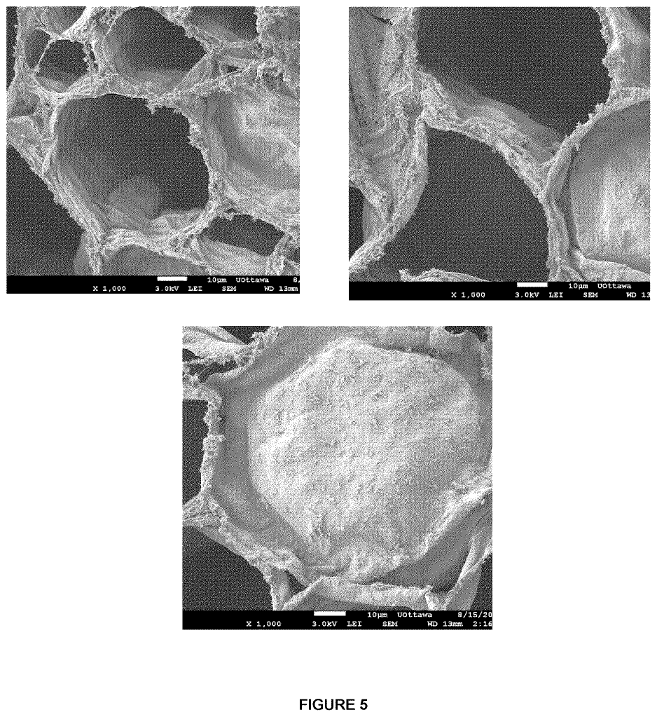

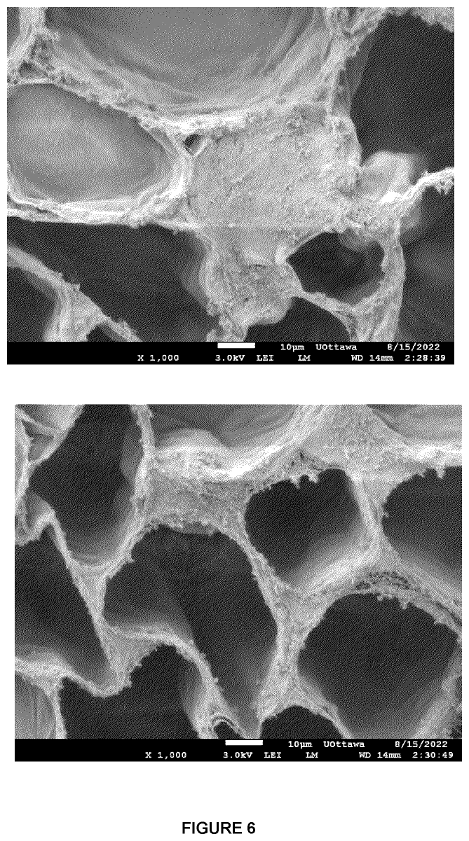

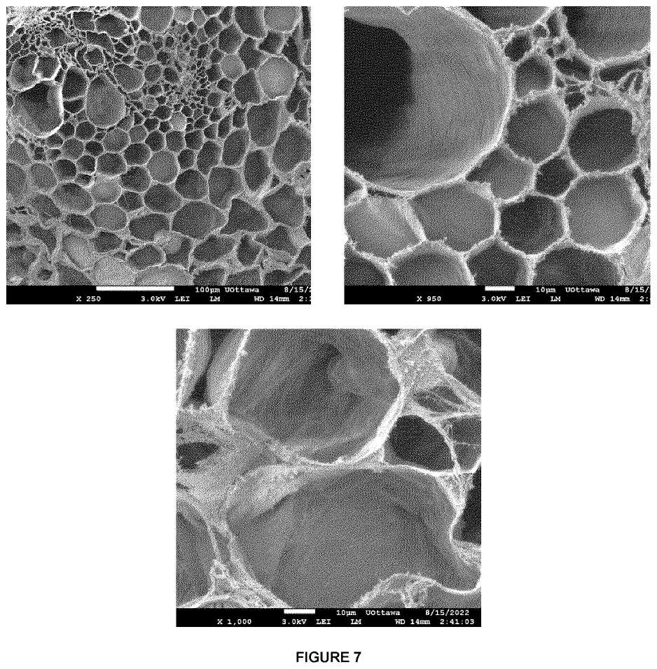

Additional microscopy data is provided in appended figures. E.g. In FIG. 5 in-vitro SEM of 20 μg/ml PLO coated asparagus scaffold, in FIG. 6 in-vitro SEM of 40 μg/ml PLO coated asparagus scaffold is shown, and in FIG. 7 in-vitro SEM of 100 g/ml PLO coated asparagus scaffold is shown.

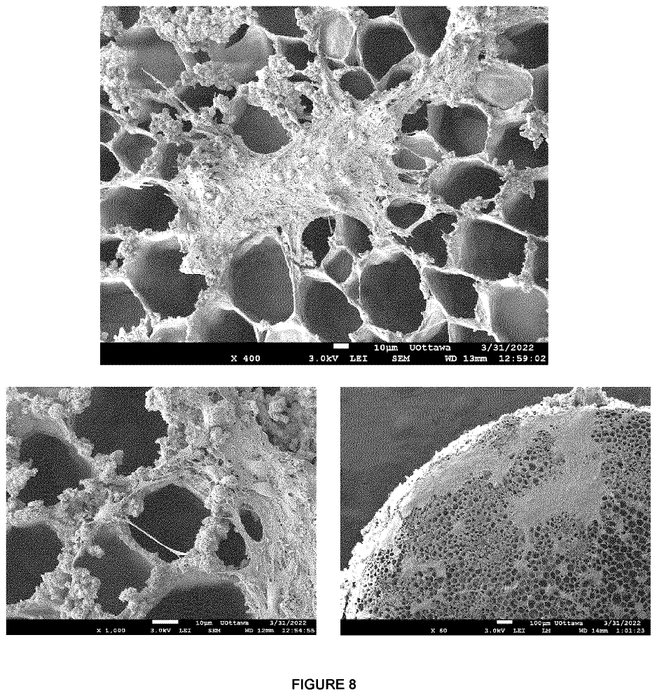

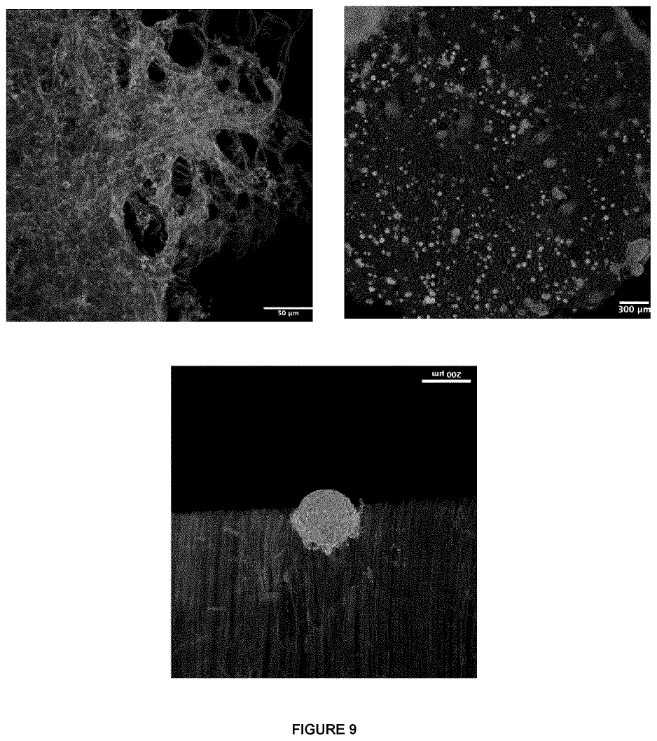

In FIG. 8, 20 μg/ml PLO coated asparagus scaffold seeded with fetal rat neural stem cells is shown. In FIG. 9 in vitro cell culture of fetal rat neural stem cells confocal microscopy (maximum intensity projection) is shown. F-actin was stained with Fluorescein Phalloidin, Nuclei were stained with Hoechst 33342 (blue) and cellulose was stained with Congo Red.

In FIG. 10 in vitro cell culture rat adult hippocampal neural stem cells confocal microscopy (maximum intensity projection) is shown. F-actin was stained with Fluorescein Phalloidin, Nuclei were stained with Hoechst 33342 (blue) and cellulose was stained with Congo Red.

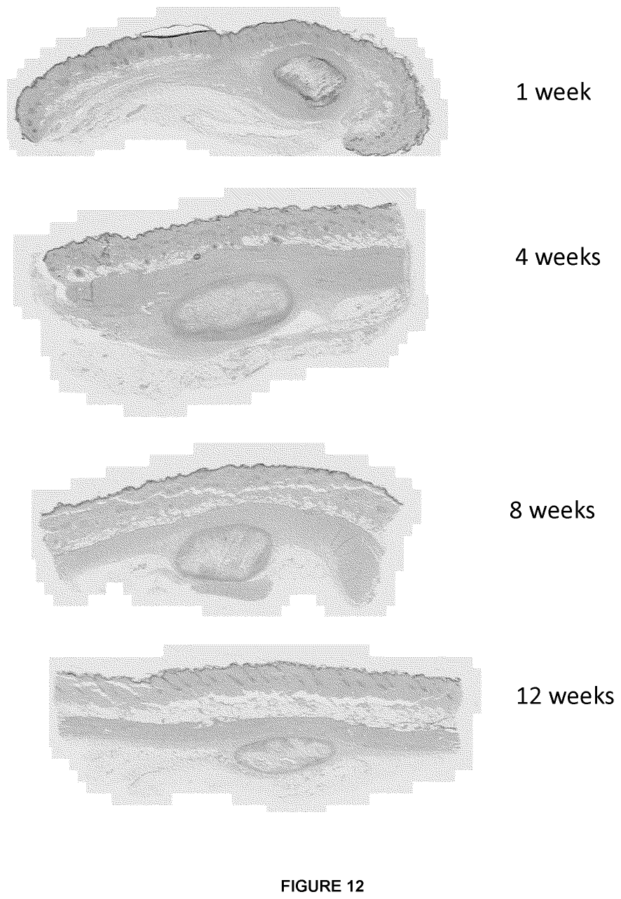

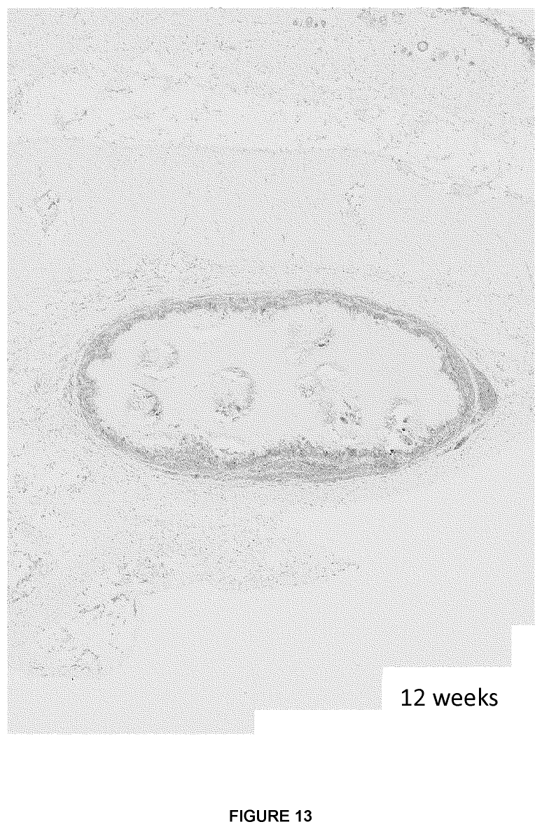

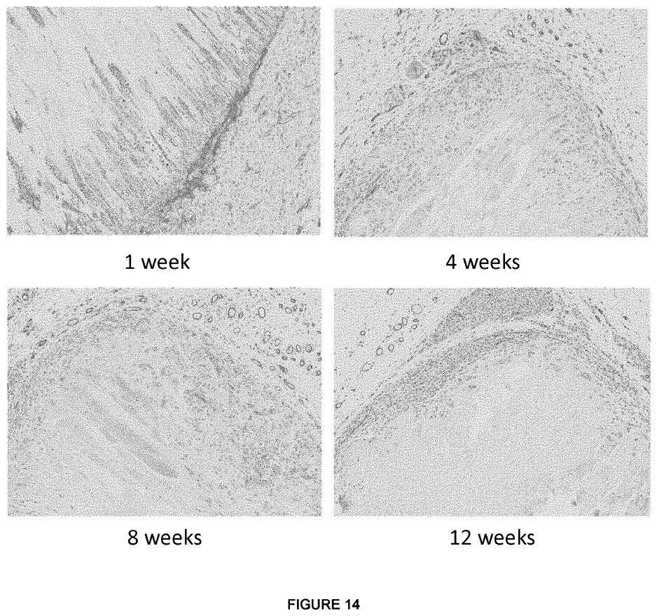

The in-vivo subcutaneous implants are shown in FIGS. 12-14. In FIG. 12 in-vivo subcutaneous implant (20 μg/ml PLO) H&E stained showing cell infiltration into the scaffolds 1 week, 4 weeks, 8 weeks and 12 weeks is shown. In FIG. 13 in vivo subcutaneous implant (20 μg/ml PLO) stained for CD45 showing minimal foreign body response at 12 weeks is shown. In FIG. 14 in-vivo subcutaneous implant (20 μg/ml PLO) stained for CD31 demonstrating that the scaffold becomes vascularized at 1 week, 4 weeks, 8 weeks and 12 weeks is shown.

The inventors also experimented with Matrigel to compare attachment of NSC's with PLO-coated scaffolds. The following steps were carried out by the inventors:
a) The matrigel was taken out of −20 °C storage and placed into an ice bucket in the 4 °C fridge to thaw overnight;
b) The following day, the pipette tips, SFM and culture dish were pre-chilled;
c) The matrigel was kept on ice at all times, sprayed with ethanol and matrigel was brought into a biological safety cabinet (BSC) on ice. The vial was swirled to mix properly.
D) 0.5ml aliquots were made from 10 ml bottle;
e) Pre-chilled pipettes and cold media was used to dilute matrigel 1:25 in SFM (to make thin coating) (1:25 is 40 μL matrigel per 1 ml SFM, 240 μL matrigel+6 ml SFM);
f) The diluted Matrigel was then added to the prechilled culture dish and the entire surface was covered;
g) This was followed by an incubation step at room temperature for 1 hour;
h) The unbound Matrigel was aspirated and rinsed gently with SFM; and
i) The matrigel was used immediately or kept at 4 °C until ~1 h before use.

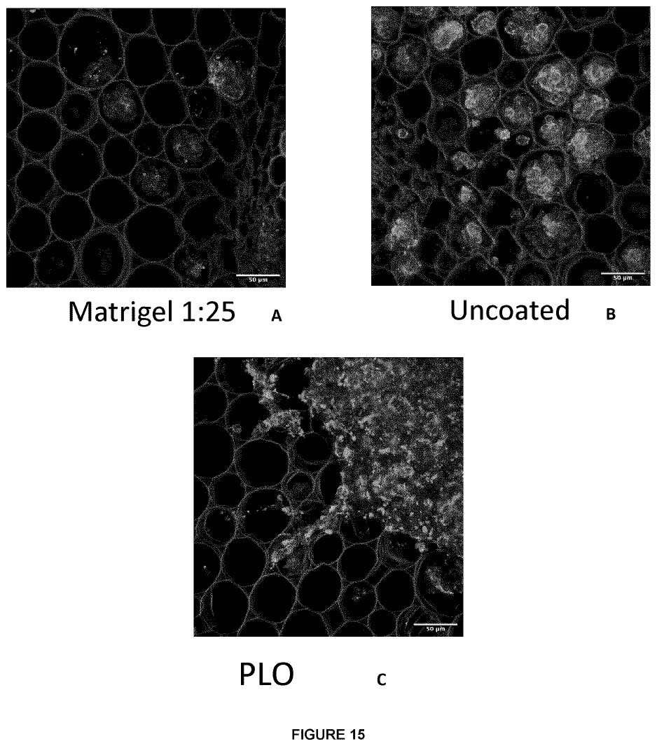

As can be seen in FIG. 15, the attachment of NSC's to matrigel visible but is not optimal. In FIG. 15A attachment to Matrigel 1:25 is shown, in FIG. 15B attachment to Uncoated Scaffold is shown, and in FIG. 15C attachment to PLO coated scaffold is shown.

### 2.1. Cellulose Scaffold Production and Implantation in Rodent Spinal Cord Injury

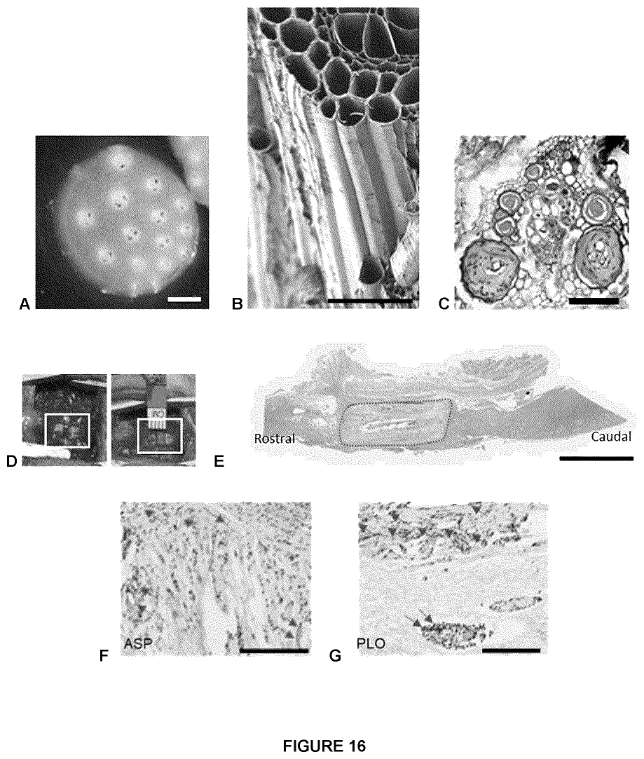

Stalks of Asparagus officinalis were cut into cylindrical sections with a biopsy punch and decellularized to produce cellulose scaffolds (FIG. 16A). Naturally occurring vascular bundles (VBs) cross the length of the cellulose scaffold, which has parenchyma with an average pore diameter of 39±15 μm (FIG. 16B). The aligned channels within the scaffold were characterized by scanning electron microscopy and the Young's modulus was measured parallel to the long axis as 128±20 kPa (n=5). In animals with a complete T8-T9 spinal cord transection, scaffolds were implanted into the gap between the stumps of the cord with the long axis of the VBs parallel to the spinal cord (FIG. 16D). The control group (n=8) did not receive a scaffold, while the PLO group (n=11) was implanted with a cellulose scaffold coated with a 100 μg/ml solution of poly-L-ornithine (PLO) and the ASP group (n=9) received an uncoated cellulose scaffold. After 12 weeks in vivo, spinal cord tissue was collected, and hematoxylin & eosin staining was performed to assess tissue integration with the scaffold (FIG. 16E). Nuclei were observed to be migrating along the channels of the cellulose biomaterial, especially within the vascular bundles (FIG. 16C), and completely crossed the lesion. The scaffolds retained their structure and dimensions throughout the study and no signs of foreign body reaction were observed. Immunohistochemistry staining for CD31 was performed to assess biomaterial vascularization (FIGS. 16F and 16G). CD31 positive blood vessels were identified in the tissue surrounding the scaffold and inside the vascular bundles within the biomaterial.

FIG. 16 shows plant-derived cellulose biomaterial in rodent model of complete transection spinal cord injury. FIG. 16A shows decellularized plant-derived scaffold consisting of vascular bundles and parenchyma (scale bar=1 mm). FIG. 16B shows SEM of cellulose scaffold microarchitecture (scale bar=100 μm). FIG. 16C shows hematoxylin & eosin staining of cross section of cellulose scaffold after 12 weeks in vivo. As shown in the figure, the scaffold's vascular bundles are infiltrated with host cells. (Scale bar=200 μm). FIG. 16D shows an exposed fully transected spinal cord (left box) and a scaffold after implantation (right box). FIG. 16E shows hematoxylin & eosin staining of sagittal section of spinal cord tissue with cellulose scaffold (outlined) after 12 weeks in vivo (Scale bar=2 mm). FIG. 16F shows immunohistochemistry staining for CD31 (brown) in a sagittal section at the biomaterial-tissue interface (representative image from the ASP group). CD31 positive blood vessels are denoted by arrow (Scale bar=200 μm). FIG. 16G shows immunohistochemistry staining for CD31 (brown) in a sagittal section at the biomaterial-tissue interface (representative image from the PLO group). CD31 positive blood vessels are denoted by arrow (Scale bar=200 μm).

### 2.2. Hindlimb Locomotor Recovery After Complete Sci

### 2.2.1. Bbb Locomotor Assessment

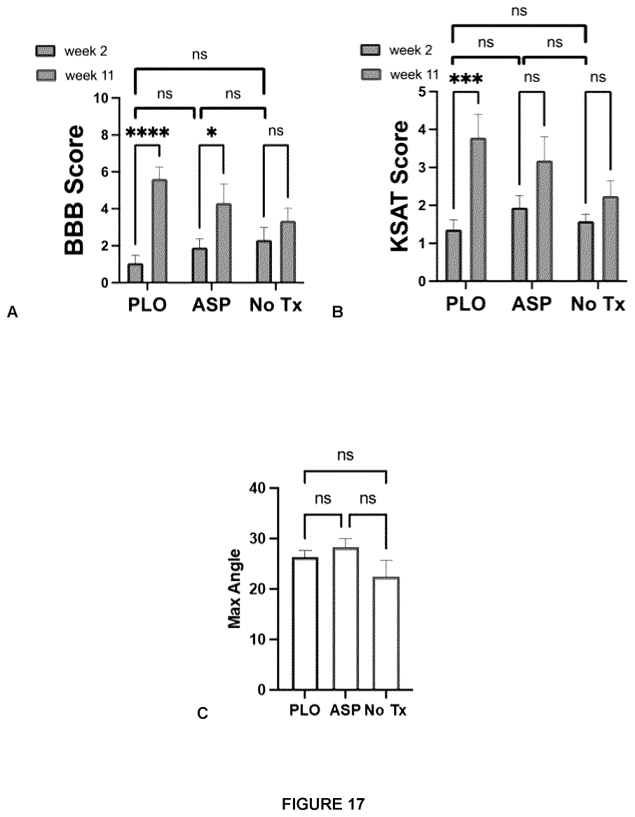

FIG. 17 shows locomotor assessments after complete spinal cord transection. FIG. 17A shows average BBB score of each experimental group at week 2 after SCI compared to week 11 after SCI (2way ANOVA Uncorrected Fisher's LSD; ****P<0.0001, *P=0.0158; mean±s.e.m, n=11 PLO animals, n=9 ASP animals, n=8 No Tx animals). FIG. 17B shows average KSAT swim assessment score of each experimental group at week 2 after SCI compared to week 11 after SCI (2way ANOVA Uncorrected Fisher's LSD; ***P=0.0001; mean±s.e.m, n=11 PLO animals, n=9 ASP animals, n=8 No Tx animals). FIG. 17C shows average maximum angle achieved by each experimental group in inclined plane assessment at final week (mean±s.e.m, n=11 PLO animals, n=9 ASP animals, n=8 No Tx animals).

Hindlimb motor function was assessed weekly using the Basso, Beattie and Bresnahan (BBB) locomotor scale (FIG. 17A). Two weeks after complete transection of the cord, the BBB assessment was performed to confirm total hindlimb paralysis and a single animal was excluded based on their score of above 5. At this first post-op BBB assessment, there were no differences amongst the experimental groups. Over the 11-week recovery period, animals that received scaffolds with and without PLO coating exhibited significant functional recovery whereas no significant change was found in BBB scores of those without scaffolds. This was determined by comparing the average score of at week 2 with the average score at week 11 for each experimental group and performing a 2way ANOVA and uncorrected Fisher's LSD test. At the final week, the mean BBB score of rats treated with the PLO-coated scaffold was 5.6±0.87 points, corresponding to slight movement in 2 joints and extensive of a third. The group receiving uncoated scaffolds had a mean score of 4.3±0.96, indicating slight movement of all three joints of the hindlimb. By contrast, controls that did not receive a scaffold had a mean score of 3.35±1.0, reflecting movement in only 2 joints. Importantly, no animals from the control group attained scores of 6 or above, whereas several animals from the PLO and ASP groups achieved BBB scores of 7 (extensive movement of all three joints), 8 (plantar placement of the paw without weight support), and 9 (plantar placement of the paw with weight support in stance only).

### 2.2.2. KSAT Swimming Assessment

During swimming, the animals rely on buoyancy to support their bodyweight, allowing for movement of unloaded limbs. As such, there is an important reduction in proprioceptive and cutaneous feedback from the hindlimbs compared to overground walking. Therefore, the Karolinska Institutet Swim Assessment Tool (KSAT) was used to identify changes in hindlimb motor ability after SCI while minimizing the influence of afferent feedback. In this assessment, healthy animals achieve a maximum score of 19, based on intensity and frequency of limb and tail movement. At this first post-op swim assessment, there were no differences amongst the experimental groups. After 11 weeks of recovery, rats with the PLO-coated scaffold showed significant improvements in swimming performance compared to their first post-op assessment whereas the controls did not achieve the same improvement (FIG. 17B). The average KSAT score of rats implanted with PLO-coated scaffolds was 3.79±0.58 compared to 3.18±0.64 with the uncoated scaffold and 2.25±0.68 for those without scaffolds. Scaffold-treated animals showed improvement primarily in hindlimb movement and trunk stability.

### 2.2.3. Inclined Plane Test

Before the spinal cord injury, all animals were trained to perform the inclined plane test. Animals were placed on an inclined plane and the slope was adjusted to determine the maximum angle at which the animal could maintain its position without falling. Every 2 weeks after the spinal cord injury, the inclined plane test was performed to examine sensorimotor recovery. No significant differences were found in the recovery of each experimental group at the final week (FIG. 17C).

### 2.2.4. 5HT Staining

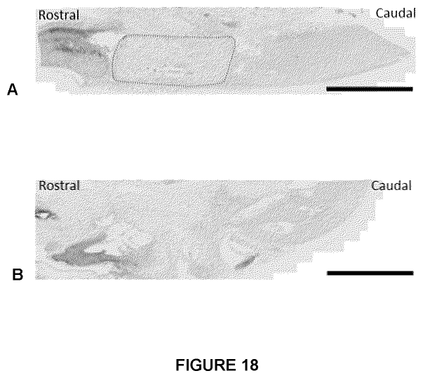

FIG. 18 shows 5-HT immunohistochemistry staining of sagittal section of T8-T9 spinal cord injury. FIG. 18A shows 5-HT staining of spinal cord implanted with ASP scaffold (as outlined). IHC was performed using DAB as the chromogen (brown) (Scale bar=2 mm). FIG. 17B shows 5-HT staining of spinal cord with no implant. IHC was performed using DAB as the chromogen (brown) (Scale bar=2mm).

Immunohistochemistry staining for 5-HT revealed some sprouting of serotonergic axons rostral to the injury (FIG. 18). However, the tissue caudal to the complete transection was devoid of 5-HT staining due to degeneration of serotonergic activity.

### 2.3. Retrograde Tract Tracing of Ascending Sensory Fibers Reveals Enhanced Regeneration

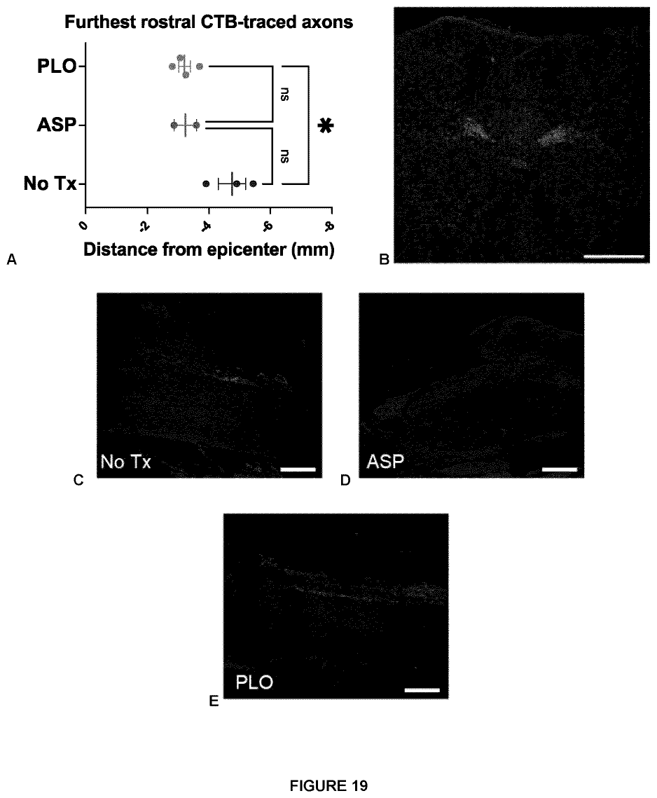

FIG. 19 shows retrograde tract tracing of ascending sensory afferents by CTb injection into sciatic nerve. FIG. 19A shows furthest rostral CTb-traced axons. Each point represents the distance (mm) between the injury epicenter and the furthest rostral CTB-traced axon in one animal. Bar represents the mean±s.e. m of each group (One-way ANOVA; *P=0.0264, n=4 PLO animals, n=2 ASP animals, n=3 No Tx animals). FIG. 19B shows hoescht (blue) stained cross section of spinal cord at T10 (caudal to the injury) confirms presence of CTb (red) in dorsal column and lamina 4 (Scale bar 300 μm). FIG. 19C shows Hoescht (blue) stained sagittal section of spinal cord from SCI animal with no scaffold. CTb-traced axons (red) can be seen in the dorsal column (Scale bar 500 μm). FIG. 19D shows Hoescht (blue) stained sagittal section of spinal cord from SCI animal with uncoated scaffold. CTb-traced axons (red) can be seen in the dorsal column (Scale bar 500 μm). FIG. 19E shows Hoescht (blue) stained sagittal section of spinal cord from SCI animal implanted with PLO-coated cellulose scaffold. CTb-traced axons (red) can be seen in the dorsal column (Scale bar 500 μm).

After 11 weeks of recovery, neuroanatomical tract tracing was performed on rats from each experimental group to identify sprouting and regenerated fibers in the injury site. For retrograde tracing, Cholera Toxin subunit B (CTb) conjugated to Alexa Fluor 647 was injected into the sciatic nerve to label sensory axons projecting along the dorsal column of the spinal cord. Cross sections of T10 spinal cord tissue caudal to the injury were imaged by confocal laser scanning microscopy and revealed a distinct CTb-positive signal in the dorsal column (FIG. 19B) which was not found in T6 sections cranial to the injury. Sagittal sections of spinal cord tissue showed CTb-labelled axons projecting into the caudal side of the scaffold towards the injury epicenter (FIG. 19C, D & E). Compared to animals without a scaffold, those with PLO-coated scaffolds had on average a significantly smaller distance between the furthest rostral CTb-traced axons and the injury epicenter (FIG. 19A), which may indicate a reduction in axonal retraction or enhanced regeneration. In animals treated with PLO-coated scaffolds (FIG. 19E), the furthest rostral CTb-labelled axons were identified at an average of 3.2 mm from the injury epicenter. By contrast, control animals that did not receive a scaffold had CTb-traced axons at an average distance of 4.8 mm from the epicenter (FIG. 19C).

### 2.4. Anterograde Labeling of the Corticospinal Tract

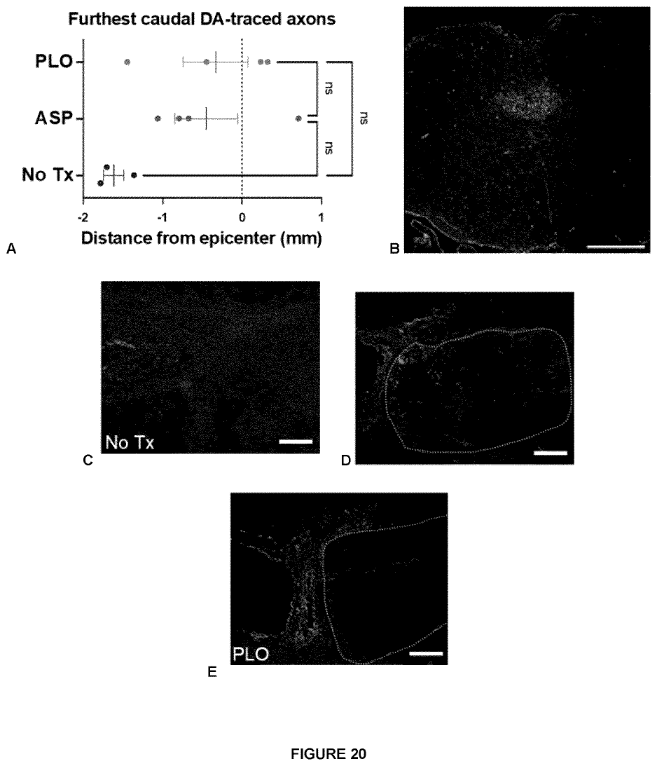

FIG. 20 shows anterograde labeling of the corticospinal tract by injection of dextran amine into hindlimb motor cortex. FIG. 20A shows furthest caudal dextran amine-traced axons. Each point represents the distance (mm) between the injury epicenter and the furthest caudal DA-traced axon in one animal. Bar represents the mean±s.e.m of each group (One-way ANOVA; P=0.0903, n=4 PLO animals, n=4 ASP animals, n=3 No Tx animals). FIG. 20B shows hoescht (blue) stained cross section of spinal cord at T6 (rostral to the injury) confirms presence of dextran amine (green) in corticospinal tract (Scale bar 300 μm). FIG. 20C shows hoescht (blue) stained sagittal section of spinal cord from SCI animal with no scaffold. DA-traced axons (green) can be seen in the corticospinal tract (Scale bar 500 μm). FIG. 20D shows hoescht (blue) stained sagittal section of spinal cord from SCI animal implanted with uncoated cellulose scaffold. DA-traced axons (green) can be seen in the corticospinal tract (Scale bar 500 μm). FIG. 20E shows hoescht (blue) stained sagittal section of spinal cord from SCI animal implanted with PLO-coated cellulose scaffold. DA-traced axons (green) can be seen in the corticospinal tract (Scale bar 500 μm).

In addition, sprouting of the corticospinal tract (CST) at the site of spinal cord injury was analyzed by stereotaxic injections of dextran amine conjugated to Alexa Fluor 488 into the hindlimb motor cortex. Successful tracer uptake was confirmed by confocal laser scanning microscopy of T6 spinal cord cross sections cranial to the injury, which showed strong positive dextran amine labelling of axons in the corticospinal tract (FIG. 20B). In sagittal sections, dextran amine-labeled axons were identified at the rostral interface between the biomaterial and spinal cord (FIGS. 20D & 20E), though none were present in T10 cord sections caudal to the injury. The most caudal dextran amine-labelled axons were identified at average distance of 0.33 mm from the epicenter for animals with PLO-coated scaffolds (FIGS. 20A & 20E), 0.45 mm from the epicenter for those with the uncoated scaffold (FIG. 20D), and 1.6 mm from the epicenter for animals without a scaffold (FIG. 20C). DA-traced axons were seen projecting along the dorsal side of the PLO-coated cellulose biomaterial towards the caudal spinal cord (FIG. 20E).

### 2.5. Neural Cell Infiltration and Axonal Sprouting Inside Cellulose Biomaterial

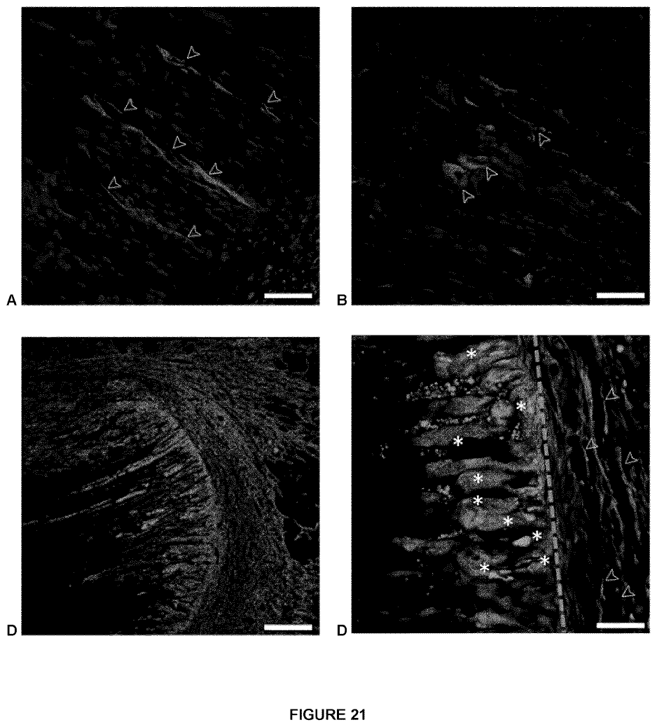

FIG. 21 shows immunostaining for β-III tubulin and neurofilament-200 reveals neural cells attaching to scaffold and migrating along channels. FIG. 21A shows β-III tubulin (red) and Hoescht (blue) staining of sagittal section within PLO-coated biomaterial. Cell bodies (identified by arrow) can be seen inside the scaffold (scale bar 50 μm). FIG. 21B β-III tubulin (red) and Hoescht (blue) staining of sagittal section within PLO-coated biomaterial. Axons (identified by arrow) can be seen sprouting inside the scaffold (scale bar 50 μm). FIG. 21C shows neurofilament 200 (green) and Hoescht (blue) staining of sagittal section at the interface (dashed line) between PLO-coated biomaterial and spinal cord (scale bar 200 μm). FIG. 21D shows Neurofilament 200 (green) and Hoescht (blue) staining of sagittal section at the interface (dashed line) between PLO-coated biomaterial and spinal cord. NF200 positive cells can be seen infiltrating the biomaterial (asterisk) & NF200 axon projections extend from the dorsal to ventral aspect of the biomaterial (arrow) (scale bar 50 μm).

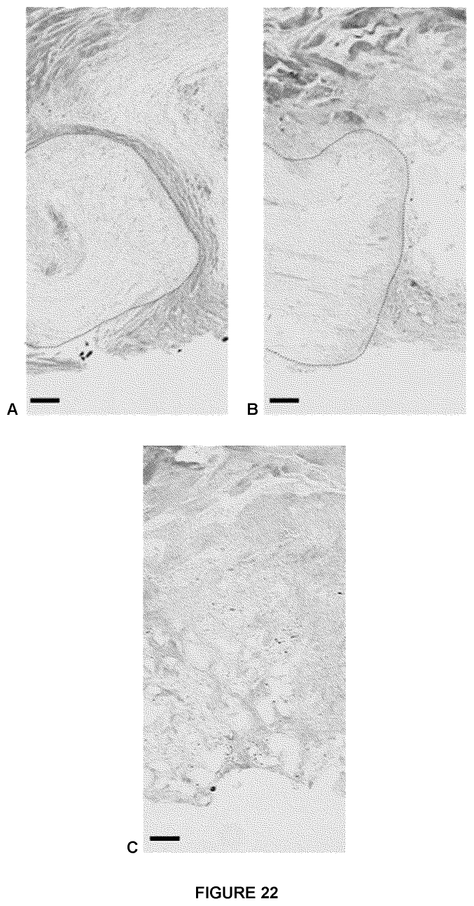

FIG. 22 shows luxol fast blue (LFB) staining of sagittal tissue sections for assessment of myelin at the site of spinal cord injury. FIG. 22A shows representative LFB staining at scaffold-tissue interface in animals implanted with PLO coated cellulose scaffold. Outline denotes cellulose scaffold (Scale bar=200 μm). FIG. 22B shows LFB staining at scaffold-tissue interface in animals implanted with uncoated cellulose scaffold (outlined) (Scale bar=200 μm). FIG. 22C shows LFB staining at cyst-tissue interface in animals with no scaffold (Scale bar=200 μm).

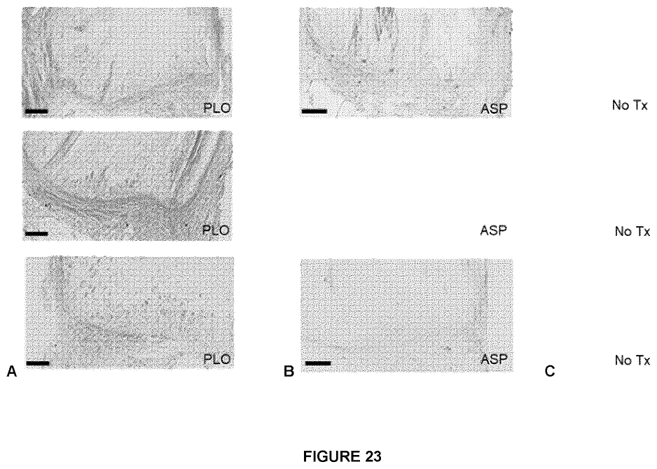

FIG. 23 shows luxol fast blue (LFB) staining of sagittal tissue sections for assessment of myelin at the site of spinal cord injury. FIG. 23A shows representative LFB staining at scaffold-tissue interface in animals implanted with PLO coated cellulose scaffold. Outline denotes cellulose scaffold (Scale bar=200 μm). FIG. 23B shows LFB staining at scaffold-tissue interface in animals implanted with uncoated cellulose scaffold (outlined) (Scale bar=200 μm). FIG. 23C shows LFB staining at cyst-tissue interface in animals with no scaffold (Scale bar=200 μm).

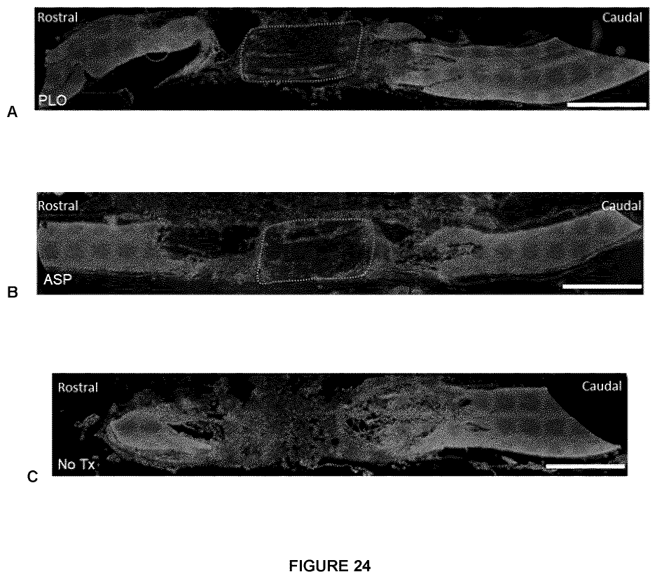

FIG. 24 shows immunostaining for glial fibrillary acidic protein (GFAP) in sagittal spinal cord sections after SCI. FIG. 24A shows GFAP (green) stained cord section from animal implanted with PLO coated cellulose scaffold. Nuclei stained with DAPI (blue). Outline denotes cellulose scaffold (Scale bar=200 μm). FIG. 24B shows GFAP (green) stained cord section from animal implanted with uncoated cellulose scaffold (outlined). Nuclei stained with DAPI (blue). (Scale bar=200 μm). FIG. 24C shows GFAP (green) stained cord section from animal with no scaffold. Nuclei stained with dapi (blue) (Scale bar=200 μm).

Histological analysis of spinal cord tissue revealed host cell infiltration into the scaffolds from the rostral and caudal interface. Within the scaffold, both cell bodies and axonal projections expressing β-III tubulin, an early neuronal marker, were identified (FIG. 21A & B). This finding suggests that endogenous adult neural stem cells infiltrated the biomaterial and initiated differentiation towards neural lineages. In addition, a substantial number of NF200-positive axons were observed inside the scaffold and surrounding tissue (FIG. 21C). Interestingly, at the rostral and caudal interfaces of the biomaterial we observed a cluster of neurofilament-positive projections branching from the dorsal to ventral aspect of the spinal cord, perpendicular to the axis of the tracts (FIG. 21D). Finally, tissue sections from each experimental group were stained with luxol fast blue (LFB) to evaluate axon myelination in the injury site (FIG. 22, FIG. 23, FIG. 24). As expected with a traumatic spinal cord injury, severe degeneration of myelin was observed in the perilesional tissue, consistent with Wallerian degeneration. However, in animals that received a PLO-coated scaffold, dark blue bands of LFB staining were observed at the rostral and caudal interface of the PLO-coated scaffold and tissue, indicating possible remyelination induced by the poly-L-ornithine (FIG. 22A-22C, 23A-23C). Immunostaining for glial fibrillary acidic protein (GFAP) in sagittal spinal cord sections after SCI is shown in FIG. 24. In FIG. 24A, GFAP (green) stained cord section is from animal implanted with PLO coated cellulose scaffold and nuclei is stained with DAPI (blue), where the denotes cellulose scaffold (Scale bar=200 μm). In FIG. 24B, GFAP (green) stained cord section is from animal implanted with uncoated cellulose scaffold (outlined) and nuclei is stained with DAPI (blue), with scale bar=200 μm. In FIG. 24C, GFAP (green) stained cord section is from animal with no scaffold and nuclei is stained with DAPI (blue), where the scale bar=200 μm.

### Discussion

Complete SCI leads to the loss of both sensorimotor and autonomic function distal to the injury site due to the interruption of ascending and descending pathways. Regrowth of axons and restoration of appropriate synaptic connectivity after SCI remains a major medical challenge. Various therapeutic strategies have attempted to induce sprouting of the corticospinal tract or to establish relay neural circuits that can mediate functional recovery. Neural circuits in the injured spinal cord are known to exhibit plasticity after treadmill training or electrical epidural stimulation that can produce some degree of motor recovery. However, irregular synaptic connectivity after SCI can be maladaptive and lead to spasticity or neuropathic pain. In recent decades, biomaterial-based therapeutic strategies for SCI have been explored as a means to support axonal growth and deliver cells or pharmaceuticals locally to modulate the inhibitory milieu of the lesion. For instance, some groups have produced bioactive scaffolds that use cell signaling molecules to enhance axonal regrowth, myelination, and functional recovery while others have 3D printed scaffolds with microarchitectures that mimic the structures in the spinal cord to support the formation of neural relays. As well, several scaffolds have been used as carriers to deliver various types of stem cells to the injured cord to promote repair.

The inventors developed a 3D cell culture scaffold consisting of decellularized Asparagus officinalis stalks that supported attachment, proliferation, and differentiation of rat adult neural stem cells. After decellularization, the cellulose scaffold was seeded with primary neural stem cells isolated from the hippocampus of adult Fisher 344 rats. Microscopic examination of the scaffolds had revealed a system of aligned channels with various diameters, which the inventors predicted would allow for efficient transport of nutrients and oxygen.

It was demonstrated that within a few hours of seeding, NSCs attached to the uncoated scaffold, both as individual cells and neurospheres of diverse sizes (FIG. 2). Moreover, the inventors noted differences in the degree of cell spreading between PLO-coated and uncoated scaffolds. NSCs seeded on PLO-coated scaffolds also appeared to migrate out from the attached neurospheres to form an underlying monolayer of cells. This behavior can potentially be explained by the fact that PLO enhances migration by promoting filopodia formation.

Subsequent F-actin staining confirmed neurosphere attachment to the biomaterial and highlighted the migration of NSCs into the channels of the scaffold. Further, NSCs were found to proliferate within the 3D culture system, as demonstrated by an Alamar Blue assay (in FIG. 3). Taken together, this data suggests that the scaffold is biocompatible and has appropriate physical characteristics to allow for neural stem cell growth.

The behavior of cells cultured in 3D systems is often different from that in 2D, including the rate of proliferation. Interestingly, the percent reduction in AlamarBlue reagent was consistently lower in 3D compared to the 2D monolayer culture system, suggesting a slight inhibition of NSC growth on the scaffolds. This difference can be attributed in part to the increased heterogeneity in the 3D culture system, where NSCs assembled into neurospheres attached to the scaffold. While the outer surface of these neurospheres consists of cells with high rates of proliferation, the inner layers of NSCs tend to be quiescent or necrotic due to reduced access to oxygen, nutrients, and growth factors. Therefore, this difference in reduction of AlamarBlue may have resulted from heterogeneity rather than purely from differences in the rate proliferation.

Finally, NSCs grown on the scaffolds were cultured in differentiation media and their expression of cytosolic markers GFAP & BIII-tubulin was evaluated after 7 days. Remarkably, this plant-derived scaffold appeared to have enhanced NSC differentiation towards neuronal cells and astrocytes. The increase in GFAP+ and βIII-tubulin+ cells on the scaffold compared to 2D controls is likely due to a complex interplay of chemical and topographical cues provided by the scaffold.

Further, mechanical tests were performed to determine the elastic modulus of the scaffold, since stiffness is one of the most important mechanical features in directing cell fate. The Young's modulus of the scaffold was determined to be 430±139 kPa, which is softer than polystyrene cell culture plates (E=3730000 kPa) but stiffer than brain and spinal cord tissue. Previous studies have shown that softer growth substrates tend to favor neuronal differentiation. Therefore, the difference in stiffness between the scaffold and the cell culture plate likely contributed to the increase in neuronal differentiation. Likewise, the scaffold's anisotropy may also be capable of modulating differentiation towards higher neurogenesis, as has been previously reported.

In a further study, the inventors investigated the ability of naturally occurring microarchitectures found in plant-derived scaffolds to provide physical support to regenerating axons and guide their extension across a lesion in the spinal cord. The results show that plant cellulose can become vascularized after implantation into the spinal cord and that endogenous neural cells expressing β-III tubulin and NF200 readily migrate into the channels of the scaffold in a linear orientation along the rostral-to-caudal axis. Aside from directing the extension of regenerating axons, the biomaterial may have contributed to motor recovery by its interaction with astrocytes, which are known to play a key role in SCI pathology. After SCI, activated microglia induce changes in the gene expression and morphology of astrocytes surrounding the lesion. The process of reactive astrogliosis is controlled by complex signaling pathways, notably NFkB or TGF-β signaling, and eventually leads to the formation of a dense glial scar. By occupying space in the injury site, the cellulose scaffold may have reduced the number of reactive astrocytes migrating into the lesion and interfered with signaling mechanisms involved in the propagation of reactive astrogliosis, thereby attenuating tissue damage and enhancing motor ability. In addition, the presence of channels at the surface of the cellulose scaffolds may have affected the alignment and morphology of astrocytes in the injury site, as previous reports have shown that cytoskeletal modulation in astrocytes can be induced by microgrooves.

In a further study, the inventors also tested the ability of plant-derived cellulose scaffolds to support repair in combination with a coating of poly-L-ornithine, a positively charged synthetic amino acid that adheres to cellulose by electrostatic interactions. PLO is commonly used in neural stem cell culture and previous studies have demonstrated its ability to improve cell attachment and migration. Specifically, PLO was shown to promote filopodia formation in neural progenitors in vitro by increasing the expression of α-Actinins 4, a critical effector of structural plasticity. Based on this, we hypothesized that PLO may have a similar effect in vivo after SCI and that increased sprouting of severed axons could assist in establishing neural relays across the scaffold, thereby mediating functional recovery.

It was found that animals implanted with the uncoated cellulose scaffold had a modest recovery of motor function in the BBB assessment and this recovery was enhanced by the addition of the poly-L-ornithine coating. To assess axon repair after complete transection, the inventors performed retrograde tract tracing with CTb, which was injected into the sciatic nerve. This neural tract tracing experiment revealed sprouting and regeneration of sensory axons in animals that received PLO-coated scaffolds. Importantly, we found that axons of the dorsal columns extended a greater distance into the injury site in animals with PLO-coated scaffolds compared to those without a scaffold. Our findings are consistent with those of Schackel et al, who found that PLO/laminin-coated hydrogels implanted in animals with a cervical hemisection had increased host cell migration and a slight increase in neurite growth. The improvements in sensory fiber regeneration that were identified in PLO-treated animals could possibly have contributed to their recovery in motor ability, given the importance of sensory feedback in motor function after SCI.

In addition to sprouting of sensory axons, the inventors found enhanced myelination in the injury site of animals with PLO-coated scaffolds, as evidenced by the luxol fast blue staining showing myelin in the scaffold periphery. In a recent study, the ability of PLO to enhance myelin repair was demonstrated in an animal model of focal demyelination. We speculate that myelin repair may be a contributing mechanism underlying PLO-induced motor recovery in our SCI model. Further, the positive effect of PLO on myelination in the injury site may be two-fold, as myelin breakdown products are known to be inhibitors of neuronal plasticity. Hence, by enhancing remyelination, PLO may also be creating a more permissive environment for axonal sprouting, consistent with our observations.

Environmental enrichment (EE) was also included as part of the multifaceted neurorehabilitation strategy due to its ability to enhance neuroplasticity and attenuate SCI-induced neuropathic pain. Environmental enrichment is a housing manipulation that includes novel toys, tunnels, nesting materials, puzzles, and running wheels along with opportunities for socialization with conspecifics. By enhancing plasticity, enriched environments support functional improvements in animal models of stroke and SCI. Studies have indicated that EE promotes plasticity through various mechanisms including increased production of neurotrophic factors and changes in dendritic-spine density. In rodent SCI models, enrichment potentiates the regenerative ability of neurons via Creb-binding protein-mediated histone acetylation, which increases expression of regeneration-associated genes. Many studies in the past have reported that environmental enrichment substantially improves sensory and motor recovery after contusive SCI. Similarly, in animal models of stroke, exposure to an enriched environment causes neuroanatomical changes including dendritic remodeling, axonal sprouting, and the release of growth factors. It has also been reported that environmental enrichment may promote white matter recovery after stroke by reducing microglia activation. Altogether, environmental enrichment shows great promise as part of a multimodal SCI therapeutic strategy.

In summary, the inventors demonstrated that cellulose scaffolds can support growth and differentiation of neural stem cells in vitro. These findings suggest that plant derived scaffolds could facilitate the production or growth of large numbers of specifically differentiated cells needed for NSC research or regenerative medicine.

The inventors also demonstrated the potential of plant-derived cellulose scaffolds to be functionalized with peptides and used in a multimodal SCI therapeutic strategy that includes environmental enrichment. Specifically, it was found that coating the biomaterial with poly-L-ornithine supported hindlimb motor recovery and neural tissue repair in a rat model of complete transection. The biomaterial was infiltrated by endogenous neural cells, which migrated along the linearly oriented channels of the plant scaffold. Furthermore, retrograde neural tracing highlighted regeneration of sensory tracts in the spinal cord after treatment with PLO-coated scaffolds. Overall, our results point to exciting potential patient-treatment strategies that use plant-derived scaffolds in combination with other therapeutics.

### Description of Methods

Biomaterial Production: Asparagus scaffolds were prepared as described by Modulevsky et al. Using a 4 mm biopsy punch, Asparagus officinalis sections were cut and placed into a 50 ml Falcon tube containing 0.1% sodium dodecyl sulphate (SDS) (Sigma-Aldrich). Samples were shaken for 72 hours at 180 RPM at room temperature. The resulting cellulose scaffolds were then transferred into new sterile microcentrifuge tubes, washed and incubated for 12 hours in phosphate-buffered saline (PBS). Following the PBS washing steps, the asparagus were then incubated in 100 mM CaCl₂ for 24 hours at room temperature and washed 3 times with dH₂O. Samples were then sterilized in 70% ethanol overnight. Finally, they were then washed 12 times with sterile 1X PBS.

Cellulose scaffolds were prepared using a 4 mm diameter biopsy punch to cut Asparagus officinalis sections which were then placed into a 50 ml Falcon tube containing 0.1% sodium dodecyl sulphate (SDS) (Sigma-Aldrich). Samples were shaken for 72 hours at 180 RPM at room temperature. The resulting cellulose scaffolds were then transferred into new sterile microcentrifuge tubes, washed and incubated for 12 hours in phosphate-buffered saline (PBS). Following the PBS washing steps, the asparagus were then incubated in 100 mM CaCl2 for 24 hours at room temperature and washed 3 times with dH2O. Samples were then sterilized in 70% ethanol overnight. Finally, scaffolds were washed 12 times in a sterile saline solution before incubating overnight in a 100 μg/ml solution of poly-L-ornithine (30-70 kDa, Sigma-Aldrich, P3655).

Scanning Electron Microscopy: Scanning electron microscopy was performed at the 2-week timepoint. NSC-seeded scaffolds were fixed with 4% PFA and dehydrated through successive gradients of ethanol (50%, 70%, 95% and 100%). Samples were dried with a critical point dryer (SAMDRI-PVT- 3D) then gold-coated at a current of 15 mA for 3 minutes with a Hitachi E-1010 ion sputter device. SEM imaging was conducted at voltages ranging from 2.00-10.0 kV on a JSM-7500F Field Emission SEM (JEOL).

Mechanical Testing: Scaffolds (4 mm diameter×1.2 mm height) were placed onto a CellScale UniVert (CellScale) compression platform for tensile testing. Each scaffold (n=15) was compressed mechanically to a maximum 10% strain, at a compression speed of 50 μm/s. The elastic modulus was determined from the slope of the linear region of the resulting stress-strain curves.

Cell culture and scaffold seeding: The resulting cellulose scaffolds were incubated in poly-L-ornithine (Sigma, 20 g/ml in dH₂O) overnight at room temperature. PLO-coated scaffolds were rinsed twice with sterile water before being transferred into a 96-well plate. Rat adult hippocampal neural stem cells (Sigma SCR 022) were cultured in serum free medium (1X KnockOut D-MEM/F-12 with 2% StemPro Neural Supplement (ThermoFisher A1050801), 20 ng/ml bFGF, 20 ng/ml EGF, and 2 mM GlutaMAX-I). Culture vessels were also coated with 20 μg/ml poly-L-ornithine and 10 μg/ml laminin as described above. An 80 μL droplet containing 200 000 cells was deposited onto every scaffold, which was then incubated at 37 °C and 5% CO2. After 4 hours of incubation, 2 mL of StemPro NSC SFM complete medium was added to each scaffold, which was then incubated for 48 hours before transferring scaffolds to a new 96-well plate. For 2-4 weeks, media was exchanged daily.

Staining and Confocal Microscopy: Cell attachment and morphology was documented by phase contrast microscopy at day 3, 7, and 14 post seeding. Staining and confocal microscopy was performed at 72 h or 14 days in culture. NSC-seeded scaffolds were fixed with warm 4% paraformaldehyde for 10 minutes then incubated 3 minutes in warm permeabilization buffer (0.5% Triton-X, 20 mM HEPES, 300 mM Sucrose, 50 mM NaCl, 3 mM MgCl2, 0.05% sodium azide). Samples were then incubated for 15 minutes in Fluorescein Phalloidin (1:100, ThermoFisher F432) to stain F-actin. Samples were rinsed with PBS and incubated in Hoechst (1:200, ThermoFisher) for 10 minutes to label nuclei. Scaffolds were incubated in 0.2% Congo Red (Sigma) for 15 minutes before a final PBS rinse. Samples were mounted in Vectashield (Vector Labs) and imaged on confocal.

Alamar Blue Cell Proliferation Assay: To measure cell proliferation, an Alamar blue assay was performed according to the manufacturer's protocol. Briefly, PLO-coated asparagus scaffolds were placed into the wells of a 96-well plate and seeded with 100 000 adult rat hippocampal neural stem cells. After 1, 2 or 5 days, cell-seeded scaffolds were transferred into a new well and 200 μL of fresh media containing 10% Alamar blue (cat. BUF 012A Bio-Rad) was added to each scaffold. After 4 hours of incubation at 37 °C, 100 μL of media was removed from each well and deposited into an empty well before reading absorbance at 570 nm and 600 nm on a spectrophotometer (Epoch 2, BioTek). Each timepoint included biological triplicates for cell-seeded scaffolds (n=3), 2D controls (NSCs grown on a PLO-coated well, n=3) and blanks (media only, n=3). Absorbances were corrected by subtracting the average absorbance of blanks at 570 nm & 600 nm.

NSC Differentiation: For neuronal and astrocyte differentiation, cells were cultured in 1X KnockOut DMEM/F-12 supplemented with 2% B27 (cat. 17504044 ThermoFisher), 2 mM GlutaMAX-I (ThermoFisher)) for 7 days.

Immunostaining for GFAP and B-tubulin: Cells or cell-seeded scaffolds were fixed in 4% paraformaldehyde for 15 minutes at room temperature. Samples were incubated for 5 minutes in permeabilization buffer (0.5% Triton-X, 20 mM HEPES, 300 mM Sucrose, 50 mM NaCl, 3 mM MgCl2, 0.05% sodium azide). After blocking in 6% Normal Goat Serum in 1X PBS for 10 minutes, samples were incubated in rabbit anti-GFAP antibody (cat. AB 5804 Sigma, 1:1000 in 1X PBS) or mouse anti-B-III tubulin antibody (cat. MAB 1195 R&D systems, 10 μg/ml in 1X PBS) overnight at 4 °C. The following day, samples were washed twice in 1xPBS (5 mins, RT) followed by a 2-hour incubation in secondary antibody: goat anti-rabbit IgG Alexa Fluor 488 (cat. A 11008 Invitrogen, 1:500 in 1X PBS) or goat anti-mouse IgG Alexa Fluor 594 (cat. A 11005 Invitrogen, 1:200 in 1X PBS). Samples were then washed in 1X PBS and counterstained with Hoescht 33342 (1: 2000 in 1X PBS) for 10 minutes.

Image analysis: For III-tubulin stains, analysis was performed on 13 images at 40X magnification for each condition (2D and 3D). Total cell number was determined by cell counting Hoescht-labeled nuclei using FIJI. Cells were considered BIII-tubulin+ when signal from red and blue channels were colocalized. For GFAP stains, analysis was performed on 6 images at 40X magnification for each condition (2D and 3D). Cells were considered GFAP+ when signal from green and blue channels were colocalized.

Animal Housing and Environmental Enrichment: All procedures described were approved and performed in accordance with standards set out by the University of Ottawa Animal Care and Veterinary Services ethical review committee. Juvenile female Sprague Dawley rats were purchased from Charles River. Rat tickling was performed with the juvenile rats as a habituation technique to improve welfare. Experimenters followed the Panksepp method of rat tickling which consists of 15 sec rest followed by 15 sec of dorsal contacts and pins for a total of 2 minutes for 4 days of training. Upon completing tickle training, every rat was tickled before every procedure. Clicker training was performed with the juvenile rats to encourage cooperation in all behavioral assays and to develop positive affect with experimenters. Briefly, rats were encouraged to step onto a plastic platform and given food rewards upon successful completion, accompanied by a ‘click’. Clicker training was done for 5 days (4 mins daily). Animals were trained to perform the inclined plane test, which consists of placing the rat on an inclined plane and adjusting the slope to determine the maximum angle at which the animal can maintain its position without falling. All rats were housed in pairs throughout the study and got 20 minutes of daily group play with conspecifics. During group play, up to 10 rats were placed in a 1-meter diameter arena with climbing structures, tunnels, foraging toys, food enrichment and nesting material. Enrichment was provided in all rats' home cages, including a wooden block, nylon bone (Bio-Serv, K3580), bunny block (Bio-Serv, F05274) and metal swing. In addition to a diet of standard rat chow, all rats were given daily food enrichment including mini yogurt drops (Cedarlane Bio-Serve, F7577), banana chips (Cedarlane Bio-Serve F7161), fruity bites (Cedarlane Bio-Serve F6038), ABC fruit blend (Cedarlane Bio-Serve F7228), Mealworms (Cedarlane Bio-Serve 9264), veggie-bites (Cedarlane Bio-Serve F5158) and pumpkin (E.D. Smith).

Spinal Cord Transection Surgical Procedure: Once rats reach a weight of 250-300 g, they are anesthetized with isoflurane USP-PPC and injected subcutaneously with normal saline (Baxter) and enrofloxacin (Baytril). Laminectomies were performed at the T8-T9 level to expose the spinal cord, the dura was removed, and the entire cord was gently lifted by a hook before cutting with micro scissors. Surgifoam 1972 (Ethicon) was used to establish hemostatic control, then the gap was measured after 10 minutes to select the appropriately sized cellulose implant. Prior to surgery, animals were be split into four groups: sham (laminectomy only), untreated (full transection only), ASP treated (cellulose implant only), and PLO treated (PLO-coated cellulose implant). PLO-coated scaffolds were rinsed twice with sterile water before implantation. While implanting the scaffold, ARTISS fibrin sealant was be applied into the cavity. The muscle and adipose tissue are reapproximated with 3-0 Vicryl sutures (Johnson & Johnson) and the skin is closed with Michel clips (Fine Science Tools). Rats received post-operative care including bladder expressions 4 times daily, pain monitoring and management with buprenorphine HCl, when necessary, weight loss and dehydration tracking.

BBB Locomotor Assessment: Functional recovery of the hindlimbs was assessed by a weekly BBB open field assessment. Each rat was placed in a 1-meter diameter arena covered with a non-slippery floor and recorded by 5 cameras. The 4-minute videos were then scored by three blinded observers and the average of three examiners was calculated for each animal at each timepoint. Spasticity and movement occurring simultaneously with urination was ignored and confirmed with repeated views of the videos. Two weeks after receiving the spinal cord injury, one animal was excluded based on their BBB score, which was above 5.

KSAT Swim Assessment: Before SCI, all rats were acclimatized to the pool daily (up to 5 minutes) for 5 days. During this pre-training, each rat was placed into a clear acrylic swim tank (depth 20 cm, length 150 cm) filled with water (27-30 °C) and encouraged to swim via clicker training. Animals with poor performance were excluded from the study before the SCI surgery. After rats sustained a SCI, the swim assessment was performed every 2 weeks to assess functional recovery. Each animal was allowed three runs across the swim tank with 20 second rests between runs. Swimming was recorded by 4 cameras and videos were scored by 3 blind observers using the Karolinska Institutet Swim Assessment Tool (KSAT).

Retrograde Labelling of Ascending Sensory Afferents: Animals also received 2 μL of Cholera Toxin Subunit B conjugated to Alexa Fluor 647 (CTb, ThermoFisher, cat. C 34778, 1% solution in sterile PBS) injected bilaterally into their sciatic nerves (4 μL total per rat). Rats were anesthetized with 3% isoflurane, a linear incision was made along the femur, and the gluteus maximus was separated from the gluteus medius to expose the sciatic nerve. Using a surgical microscope, a nick was made in the proximal portion of the exposed nerve. The CTb solution was loaded into a Hamilton syringe, and the needle was inserted 5 mm into the sciatic nerve. CTb was injected in increments of 0.5 μL, waiting 30 seconds and withdrawing the needle by 1 mm per injection. Once the injection was complete, a sterile Q-tip was used to remove any CTb that was expelled from the nerve and the incision was sutured using 4-0 Prolene sutures. Post-operatively, 2% bupivacaine was applied topically and animals were given buprenorphine (0.05 mg/kg subcutaneously) and Gapapentin (Chiron, 50 mg/kg subcutaneously) for analgesia. Pain was assessed TID and additional buprenorphine was administered as necessary. To prevent infections, Enrofloxacin (Bayer, 10 mg/kg subcutaneously) was administered daily for 3 days peri-operatively.

Anterograde Labeling of the Corticospinal Tract: 11 weeks post-operatively, rats from each group were injected with neural tracers to visualize axons in and around the injury site. Animals were anesthetized with 3% isoflurane and placed into a stereotactic frame. After a surgical scrub, a sagittal incision was made to expose the top of the skull and the periosteum was scraped off. Using a Zeiss surgical microscope, the skull was levelled by measuring the Dorsal-Ventral axis at 4 random points and ensuring they are within 0.05 mm of each other. Bregma was located and its coordinates were used to calculate the target location of each injection. Using a surgical drill (Micro Drill, Harvard Apparatus) attached to the stereotactic frame, 8 holes were drilled into the cranium. Stereotactic injections were made into the right and left motor cortex at coordinates: Injection 1: Anterior-posterior (AP)-0.5 mm, Medial-lateral (ML)±2 mm, Dorsal-Ventral (DV) 1.5 mm. Injection 2: Anterior-posterior (AP)-1 mm, Medial-lateral (ML)±2.5 mm, Dorsal-Ventral (DV) 1.5 mm. Injection 3: Anterior-posterior (AP)-1.5 mm, Medial-lateral (ML)±2 mm, Dorsal-Ventral (DV) 1.5 mm. Injection 4: Anterior-posterior (AP)-2 mm, Medial-lateral (ML)±2.5 mm, Dorsal-Ventral (DV) 1.5 mm. Injections were made using a 5 uL Hamilton syringe. After a 2-minute delay, a volume of 0.5 μL of 10% Dextran Amine Alexa Fluor 488 (ThermoFisher, cat. D22910, 10,000 MW) was injected into each location at a rate of 250 nl/min for a total injection volume of 4 μL per rat. Each injection was followed by a 2-minute delay to ensure diffusion into the tissue. Once all 8 injections were complete, the scalp was sutured with 4-0 Prolene sutures and 2% transdermal bupivacaine was applied to the incision. Animals had 2 weeks of recovery time before euthanasia to allow for transport of neural tracers. For tissue collection, animals were deeply anesthetized with isoflurane USP-PPC and euthanized by cardiac perfusion with 500 ml of 1xPBS followed by 500ml of 4% paraformaldehyde. The brain and spinal cord were dissected out and fixed overnight in 4% paraformaldehyde at 40 □C, then stored in 70% ethanol at 40 □C until embedding and sectioning.

Quantification of neuroanatomical tract tracing: Cross-sections of spinal cord tissue above and below the injury site were imaged by confocal laser scanning microscopy to confirm successful tracer uptake. For retrograde tracing, CTb-traced axons were seen in the dorsal columns of T10 cross sections. For anterograde tracing, dextran amine labelled axons were identified within the corticospinal tract of T6 cross sections. For each animal, 3 sagittal sections in the middle of the cord were imaged by confocal laser scanning microscopy and the distance between the tracers and the injury epicenter was measured using FIJI. For retrograde tracing with CTb (n=4 PLO animals, n=2 ASP animals, n=3 No Tx animals), the distance (mm) was measured between the furthest rostral CTb-traced axon and the injury epicenter. For anterograde tracing with dextran amine (n=4 PLO animals, n=4 ASP animals, n=3 No Tx animals), the distance (mm) was measured between the furthest caudal dextran amine-traced axon and the injury epicenter. Averages were calculated for each group and a one-way ANOVA was performed.

GFAP Immunostaining: Paraformaldehyde fixed paraffin embedded tissue sections (5 μm thick) were deparaffinized and pre-treated using heat mediated antigen retrieval with Sodium Citrate buffer (pH 6.0). Slides were then rehydrated in 1X TBST buffer and blocked for 30 minutes with Rodent Block R (Biocare RBR962H). Sections were then incubated with Rabbit GFAP (1:3000, Sigma AB5804) at room temperature for 1.5 hours. Sections were washed with 1XTBST and then incubated with the Goat anti-Rabbit-488 antibody for 2 hours in the dark at room temperature. This was followed by incubation with a quencher (Vector True View Autofluorescence Quenching Kit #SP-8400, Vector Labs) to decrease autofluorescence. Sections were then washed, incubated with 5 ug/ml of DAPI (ThermoScientific #62248) and coverslipped.

β-III Tubulin Immunostaining: Paraformaldehyde fixed paraffin embedded tissue sections (5 μm thick) were deparaffinized and tissue was permeabilized at room temperature (5 mins) the following buffer: 0.5% Triton-X, 20 mM HEPES, 300 mM Sucrose, 50 mM NaCl, 3 mM MgCl2, 0.05% sodium azide. Sections were incubated in blocking buffer (6% Normal Goat Serum in 1xPBS) for 10 minutes. Sections were rinsed twice in 1xPBS before incubation at 4 °C overnight in mouse anti-β-III tubulin antibody (10 ug/ml, MAB 1195, R&D systems). The following day, after 2 washes of 1xPBS, sections were incubated at room temperature (2.5 hours) in goat anti-mouse alexa Fluor 594 polyclonal antibody (1:200, A11005, Thermofisher) and counterstained with Hoescht (1:2000).

Neurofilament 200 Immunostaining: Paraformaldehyde fixed paraffin embedded tissue sections (5 μm thick) were deparaffinized and tissue was permeabilized at room temperature (3x 5 minutes) in 1X TBST. Sections were incubated in blocking buffer (5% Normal Goat Serum in 1X TBST) for 30 minutes followed by 3 washes in 1X TBST. Tissue was incubated at 4 °C overnight in rabbit anti-NF200 (1:3000 dilution in 1X PBS, cat. N 4142 Sigma). The following day, after 2 washes of 1X PBS, sections were incubated at room temperature (2 hours) in goat anti-rabbit alexa fluor 488 (cat. A11008 Thermofisher) and counterstained with Hoescht (1:2000).

Statistical Analysis: P values were calculated using two-tailed Student's t test. Results were considered as statistically significant when P<0.01. Numerical data are expressed as mean±standard deviation. Experimental data were analyzed using GraphPad Prism. The differences between two groups were compared utilizing the t test, while those among multiple groups were compared by ANOVA.

As would be appreciated by a person skilled in the art, one or more illustrative embodiments have been described by way of example only. It will be understood to persons skilled in the art that a number of variations and modifications can be made without departing from the scope of the invention as defined in the claims.

All citations and/or references recited herein are hereby incorporated by reference in their entirety.

### REFERENCES

Gage, F. H. Mammalian Neural Stem Cells. Science (1979) 287, 1433-1438 (2000).
Tang, Y., Yu, P. & Cheng, L. Current progress in the derivation and therapeutic application of neural stem cells. Cell Death Dis 8, e3108-e3108 (2017).
Cao, Q., Benton, R. L. & Whittemore, S. R. Stem cell repair of central nervous system injury. J Neurosci Res 68, 501-510 (2002).
Zhao, X. -M. et al. Neural Stem Cell Transplantation Improves Locomotor Function in Spinal Cord Transection Rats Associated with Nerve Regeneration and IGF-1 R Expression. Cell Transplant 28, 1197-1211 (2019).
Zhu, J., Wu, X. & Zhang, H. Adult Neural Stem Cell Therapy: Expansion In Vitro, Tracking In Vivo and Clinical Transplantation. Curr Drug Targets 6, 97-110 (2005).
Tejeda, G., Ciciriello, A. J. & Dumont, C. M. Biomaterial Strategies to Bolster Neural Stem Cell-Mediated Repair of the Central Nervous System. Cells Tissues Organs 211, 655-669 (2022).
Hwang, D. W. et al. In Vivo Bioluminescence Imaging for Prolonged Survival of Transplanted Human Neural Stem Cells Using 3D Biocompatible Scaffold in Corticectomized Rat Model. PLOS One 9, e105129 (2014).
Rammensee, S., Kang, M. S., Georgiou, K., Kumar, S. & Schaffer, D. v. Dynamics of Mechanosensitive Neural Stem Cell Differentiation. Stem Cells 35, 497-506 (2017).
Saha, K. et al. Substrate Modulus Directs Neural Stem Cell Behavior. Biophys J 95, 4426-4438 (2008).
Chen, W., Shao, Y., Li, X., Zhao, G. & Fu, J. Nanotopographical surfaces for stem cell fate control: Engineering mechanobiology from the bottom. Nano Today 9, 759-784 (2014).
Lim, S. H., Liu, X. Y., Song, H., Yarema, K. J. & Mao, H. -Q. The effect of nanofiber-guided cell alignment on the preferential differentiation of neural stem cells. Biomaterials 31, 9031-9039 (2010).
Czeisler, C. et al. Surface topography during neural stem cell differentiation regulates cell migration and cell morphology. Journal of Comparative Neurology 524, 3485-3502 (2016).
Liu, F. et al. The Influence of the Surface Topographical Cues of Biomaterials on Nerve Cells in Peripheral Nerve Regeneration: A Review. Stem Cells Int 2021, 1-13 (2021).
Engler, A. J., Sen, S., Sweeney, H. L. & Discher, D. E. Matrix Elasticity Directs Stem Cell Lineage Specification. Cell 126, 677-689 (2006).
Wang, J. -H., Hung, C. -H. & Young, T. -H. Proliferation and differentiation of neural stem cells on lysine-alanine sequential polymer substrates. Biomaterials 27, 3441-3450 (2006).
Flanagan, L. A., Rebaza, L. M., Derzic, S., Schwartz, P. H. & Monuki, E. S. Regulation of human neural precursor cells by laminin and integrins. J Neurosci Res 83, 845-856 (2006).
Ren, Y. -J. et al. In vitro behavior of neural stem cells in response to different chemical functional groups. Biomaterials 30, 1036-1044 (2009).
Joseph, G., Orme, R. P., Kyriacou, T., Fricker, R. A. & Roach, P. Effects of Surface Chemistry Interaction on Primary Neural Stem Cell Neurosphere Responses. ACS Omega 6, 19901-19910 (2021).
Ge, H. et al. Poly-L-ornithine promotes preferred differentiation of neural stem/progenitor cells via ERK signalling pathway. Sci Rep 5, 15535 (2015).
Ge, H. et al. Poly-L-ornithine enhances migration of neural stem/progenitor cells via promoting α-Actinin 4 binding to actin filaments. Sci Rep 6, 37681 (2016).
Macgregor, M., Williams, R., Downes, J., Bachhuka, A. & Vasilev, K. The Role of Controlled Surface Topography and Chemistry on Mouse Embryonic Stem Cell Attachment, Growth and Self-Renewal. Materials (Basel) 10, (2017).
Edelbrock, A. N. et al. Superstructured Biomaterials Formed by Exchange Dynamics and Host-Guest Interactions in Supramolecular Polymers. Advanced Science 8, 2004042 (2021).
Fontoura, J. C. et al. Comparison of 2D and 3D cell culture models for cell growth, gene expression and drug resistance. Materials Science and Engineering: C 107, 110264 (2020).
Belfiore, L. et al. Generation and analysis of 3D cell culture models for drug discovery. European Journal of Pharmaceutical Sciences 163, 105876 (2021).
Breslin, S. & O'Driscoll, L. Three-dimensional cell culture: the missing link in drug discovery. Drug Discov Today 18, 240-249 (2013).
Hong, H. et al. Digital light processing 3D printed silk fibroin hydrogel for cartilage tissue engineering. Biomaterials 232, 119679 (2020).
Du, D., Furukawa, K. S. & Ushida, T. 3D culture of osteoblast-like cells by unidirectional or oscillatory flow for bone tissue engineering. Biotechnol Bioeng 102, 1670-1678 (2009).
Marei, I. et al. 3D Tissue-Engineered Vascular Drug Screening Platforms: Promise and Considerations. Front Cardiovasc Med 9, (2022).
Yoshii, Y. et al. High-throughput screening with nanoimprinting 3D culture for efficient drug development by mimicking the tumor environment. Biomaterials 51, 278-289 (2015).
Bao, M., Xie, J. & Huck, W. T. S. Recent Advances in Engineering the Stem Cell Microniche in 3D. Advanced Science 5, 1800448 (2018).
Edmondson, R., Broglie, J. J., Adcock, A. F. & Yang, L. Three-dimensional cell culture systems and their applications in drug discovery and cell-based biosensors. Assay Drug Dev Technol 12, 207-18 (2014).
Vagaska, B., Gillham, O. & Ferretti, P. Modelling human CNS injury with human neural stem cells in 2-and 3-Dimensional cultures. Sci Rep 10, 6785 (2020).
Hu, Y. et al. Scaffolds with anisotropic structure for neural tissue engineering. Engineered Regeneration 3, 154-162 (2022). Modulevsky, D. J., Lefebvre, C., Haase, K., Al-Rekabi, Z. & Pelling, A. E. Apple Derived Cellulose Scaffolds for 3D Mammalian Cell Culture. PLOS One 9, e97835 (2014).
Hickey, R. J., Modulevsky, D. J., Cuerrier, C. M. & Pelling, A. E. Customizing the Shape and Microenvironment Biochemistry of Biocompatible Macroscopic Plant-Derived Cellulose Scaffolds. ACS Biomater Sci Eng 4, 3726-3736 (2018).
Modulevsky, D. J., Cuerrier, C. M. & Pelling, A. E. Biocompatibility of Subcutaneously Implanted Plant-Derived Cellulose Biomaterials. PLOS One 11, e0157894 (2016).
Fontana, G. et al. Biofunctionalized Plants as Diverse Biomaterials for Human Cell Culture. Adv Healthc Mater 6, 1601225 (2017).
Indurkar, A., Pandit, A., Jain, R. & Dandekar, P. Plant-based biomaterials in tissue engineering. Bioprinting 21, e00127 (2021).
Gershlak, J. R. et al. Crossing kingdoms: Using decellularized plants as perfusable tissue engineering scaffolds. Biomaterials 125, 13-22 (2017).
Peng, W. et al. Recent progress of collagen, chitosan, alginate and other hydrogels in skin repair and wound dressing applications. Int J Biol Macromol 208, 400-408 (2022).
Zhu, T., Jiang, J., Zhao, J., Chen, S. & Yan, X. Regulating Preparation Of Functional Alginate-Chitosan Three-Dimensional Scaffold For Skin Tissue Engineering. Int J Nanomedicine Volume 14, 8891-8903 (2019).
Dikici, S., Claeyssens, F. & MacNeil, S. Decellularised baby spinach leaves and their potential use in tissue engineering applications: Studying and promoting neovascularisation. J Biomater Appl 34, 546-559 (2019).
Chen, F. et al. Natural Flammulina velutipes-Based Nerve Guidance Conduit as a Potential Biomaterial for Peripheral Nerve Regeneration: In Vitro and In Vivo Studies. ACS Biomater Sci Eng 7, 3821-3834 (2021).
Venkatesan, J. & Kim, S.-K. Chitosan Composites for Bone Tissue Engineering—An Overview. Mar Drugs 8, 2252-2266 (2010).
Maxime Leblanc Latour. Plant-derived Cellulose Scaffolds for Bone Tissue Engineering. bioRxiv (2020).
Balasundari, R. et al. Nanocoated Botanical Scaffold in Salvage for Human Tissue Regeneration. J Biomater Tissue Eng 2, 330-335 (2012).
Lee, J., Jung, H., Park, N., Park, S.-H. & Ju, J. H. Induced Osteogenesis in Plants Decellularized Scaffolds. Sci Rep 9, 20194 (2019).
Loh, Q. L. & Choong, C. Three-Dimensional Scaffolds for Tissue Engineering Applications: Role of Porosity and Pore Size. Tissue Eng Part B Rev 19, 485-502 (2013).
Rasoulianboroujeni, M. et al. Dual Porosity Protein-based Scaffolds with Enhanced Cell Infiltration and Proliferation. Sci Rep 8, 14889 (2018).
Luca, A. C. et al. Impact of the 3D Microenvironment on Phenotype, Gene Expression, and EGFR Inhibition of Colorectal Cancer Cell Lines. PLOS One 8, e59689 (2013).
Fallica, B., Maffei, J. S., Villa, S., Makin, G. & Zaman, M. Alteration of Cellular Behavior and Response to PI3K Pathway Inhibition by Culture in 3D Collagen Gels. PLOS One 7, e48024 (2012).
Oral, I., Guzel, H. & Ahmetli, G. Measuring the Young's modulus of polystyrene-based composites by tensile test and pulse-echo method. Polymer Bulletin 67, 1893-1906 (2011).
Banerjee, A. et al. The influence of hydrogel modulus on the proliferation and differentiation of encapsulated neural stem cells. Biomaterials 30, 4695-4699 (2009).
Baek, J. et al. Distinct Mechanosensing of Human Neural Stem Cells on Extremely Limited Anisotropic Cellular Contact. ACS Appl Mater Interfaces 10, 33891-33900 (2018).
Daniel J. Modulevsky et al. Plant Scaffolds Support Motor Recovery and Regeneration in Rats after Traumatic Spinal Cord Injury. bioRxiv (2022).
Zheng, B. & Tuszynski, M. H. Regulation of axonal regeneration after mammalian spinal cord injury. Nat Rev Mol Cell Biol (2023) doi: 10.1038/s41580-022-00562-y.
Johansson, C. B. et al. Identification of a Neural Stem Cell in the Adult Mammalian Central Nervous System. Cell 96, 25-34 (1999).
Lacroix, S. et al. Central Canal Ependymal Cells Proliferate Extensively in Response to Traumatic Spinal Cord Injury but Not Demyelinating Lesions. PLOS One 9, e85916 (2014).
Barnabé-Heider, F. et al. Origin of New Glial Cells in Intact and Injured Adult Spinal Cord. Cell Stem Cell 7, 470-482 (2010).
Li, J. et al. Recent advances in endogenous neural stem/progenitor cell manipulation for spinal cord injury repair. Theranostics 13, 3966-3987 (2023).
Hachem, L. D. & Fehlings, M. G. Pathophysiology of Spinal Cord Injury. Neurosurg Clin N Am 32, 305-313 (2021).
Quraishe, S., Forbes, L. H. & Andrews, M. R. The Extracellular Environment of the CNS: Influence on Plasticity, Sprouting, and Axonal Regeneration after Spinal Cord Injury. Neural Plast 2018, 1-18 (2018).
Anjum, A. et al. Spinal Cord Injury: Pathophysiology, Multimolecular Interactions, and Underlying Recovery Mechanisms. Int J Mol Sci 21, 7533 (2020).
McKerracher, L. & Rosen, K. M. MAG, myelin and overcoming growth inhibition in the CNS. Front Mol Neurosci 8, (2015).
Lee, J. K. & Zheng, B. Role of myelin-associated inhibitors in axonal repair after spinal cord injury. Exp Neurol 235, 33-42 (2012).
Chambel, S. S. & Cruz, C. D. Axonal growth inhibitors and their receptors in spinal cord injury: from biology to clinical translation. Neural Regen Res 18, 2573-2581 (2023).
Wang, J. et al. Nogo-A expression dynamically varies after spinal cord injury. Neural Regen Res 10, 225 (2015).
Pakulska, M. M., Tator, C. H. & Shoichet, M. S. Local delivery of chondroitinase ABC with or without stromal cell-derived factor 1a promotes functional repair in the injured rat spinal cord. Biomaterials 134, (2017).
Hyatt, A. J. T., Wang, D., Kwok, J. C., Fawcett, J. W. & Martin, K. R. Controlled release of chondroitinase ABC from fibrin gel reduces the level of inhibitory glycosaminoglycan chains in lesioned spinal cord. Journal of Controlled Release 147, 24-29 (2010).
Karimi-Abdolrezaee, S., Schut, D., Wang, J. & Fehlings, M. G. Chondroitinase and Growth Factors Enhance Activation and Oligodendrocyte Differentiation of Endogenous Neural Precursor Cells after Spinal Cord Injury. PLOS One 7, (2012).
Sartori, A. M., Hofer, A.-S. & Schwab, M. E. Recovery after spinal cord injury is enhanced by anti-Nogo-A antibody therapy—from animal models to clinical trials. Curr Opin Physiol 14, 1-6 (2020).
Díaz-Galindo, M. del C., Calderón-Vallejo, D., Olvera-Sandoval, C. & Quintanar, J. L. Therapeutic approaches of trophic factors in animal models and in patients with spinal cord injury. Growth Factors 38, 1-15 (2020).
Blesch, A. & Tuszynski, M. H. Cellular GDNF delivery promotes growth of motor and dorsal column sensory axons after partial and complete spinal cord transections and induces remyelination. Journal of Comparative Neurology 467, 403-417 (2003).
Bradbury, E. J., King, V. R., Simmons, L. J., Priestley, J. V. & McMahon, S. B. NT-3, but not BDNF, prevents atrophy and death of axotomized spinal cord projection neurons. European Journal of Neuroscience 10, 3058-3068 (1998).
Bregman, B. S. et al. Chapter 18 Transplants and neurotrophic factors increase regeneration and recovery of function after spinal cord injury. in 257-273 (2002). doi: 10.1016/S0079-6123(02)37020-1.
Fan, L. et al. Directing Induced Pluripotent Stem Cell Derived Neural Stem Cell Fate with a Three-Dimensional Biomimetic Hydrogel for Spinal Cord Injury Repair. ACS Appl Mater Interfaces 10, 17742-17755 (2018).
Liu, Q. et al. Electric field stimulation boosts neuronal differentiation of neural stem cells for spinal cord injury treatment via PI3K/Akt/GSK-3β/β-catenin activation. Cell Biosci 13, 4 (2023).
Sasaki, M., Lankford, K. L., Zemedkun, M. & Kocsis, J. D. Identified Olfactory Ensheathing Cells Transplanted into the Transected Dorsal Funiculus Bridge the Lesion and Form Myelin. The Journal of Neuroscience 24, 8485-8493 (2004).
Keirstead, H. S. et al. Human Embryonic Stem Cell-Derived Oligodendrocyte Progenitor Cell Transplants Remyelinate and Restore Locomotion after Spinal Cord Injury. The Journal of Neuroscience 25, 4694-4705 (2005).
Koffler, J. et al. Biomimetic 3D-printed scaffolds for spinal cord injury repair. Nat Med 25, 263-269 (2019).
Álvarez, Z. et al. Bioactive scaffolds with enhanced supramolecular motion promote recovery from spinal cord injury. Science (1979) 374, 848-856 (2021).
Pawar, K. et al. Biomaterial bridges enable regeneration and re-entry of corticospinal tract axons into the caudal spinal cord after SCI: Association with recovery of forelimb function. Biomaterials 65, 1-12 (2015).
Chedly, J. et al. Physical chitosan microhydrogels as scaffolds for spinal cord injury restoration and axon regeneration. Biomaterials 138, 91-107 (2017).
Guijarro-Belmar, A. et al. Effectiveness of biomaterial-based combination strategies for spinal cord repair—a systematic review and meta-analysis of preclinical literature. Spinal Cord 60, 1041-1049 (2022).
Meshkini, A. et al. Combined treatment with Minocycline and methylprednisolone in acute traumatic spinal cord Injury: A pilot study. Interdisciplinary Neurosurgery 36, 101883 (2024).
García, E. et al. Recent advances in the combination of cellular therapy with stem cells and nanoparticles after a spinal cord injury. Front Neurol 14, (2023).
Liu, S., Xie, Y.-Y. & Wang, B. Role and prospects of regenerative biomaterials in the repair of spinal cord injury. Neural Regen Res 14, 1352 (2019).
Shen, H. et al. Advances in Biomaterial-Based Spinal Cord Injury Repair. Adv Funct Mater 32, (2022).
Wang, J.-H., Hung, C.-H. & Young, T.-H. Proliferation and differentiation of neural stem cells on lysine-alanine sequential polymer substrates. Biomaterials 27, 3441-3450 (2006).
Flanagan, L. A., Rebaza, L. M., Derzic, S., Schwartz, P. H. & Monuki, E. S. Regulation of human neural precursor cells by laminin and integrins. J Neurosci Res 83, 845-856 (2006).
Ren, Y.-J. et al. In vitro behavior of neural stem cells in response to different chemical functional groups. Biomaterials 30, 1036-1044 (2009).
Joseph, G., Orme, R. P., Kyriacou, T., Fricker, R. A. & Roach, P. Effects of Surface Chemistry Interaction on Primary Neural Stem Cell Neurosphere Responses. ACS Omega 6, 19901-19910 (2021).
Ge, H. et al. Poly-L-ornithine promotes preferred differentiation of neural stem/progenitor cells via ERK signalling pathway. Sci Rep 5, 15535 (2015).
Kubinová, Š. & Syková, E. Nanotechnologies in regenerative medicine. Minimally Invasive Therapy & Allied Technologies 19, 144-156 (2010).
Estrada, V. et al. Long-lasting significant functional improvement in chronic severe spinal cord injury following scar resection and polyethylene glycol implantation. Neurobiol Dis 67, 165-179 (2014).
Slotkin, J. R. et al. Biodegradable scaffolds promote tissue remodeling and functional improvement in non-human primates with acute spinal cord injury. Biomaterials 123, 63-76 (2017).
Sugai, K. et al. Neural stem/progenitor cell-laden microfibers promote transplant survival in a mouse transected spinal cord injury model. J Neurosci Res 93, 1826-1838 (2015).
Breen, B. A. et al. Therapeutic Effect of Neurotrophin-3 Treatment in an Injectable Collagen Scaffold Following Rat Spinal Cord Hemisection Injury. ACS Biomater Sci Eng 3, 1287-1295 (2017).
Zou, Y. et al. Transplantation of collagen sponge-based three-dimensional neural stem cells cultured in a RCCS facilitates locomotor functional recovery in spinal cord injury animals. Biomater Sci 10, 915-924 (2022).
Li, X. et al. A collagen microchannel scaffold carrying paclitaxel-liposomes induces neuronal differentiation of neural stem cells through Wnt/β-catenin signaling for spinal cord injury repair. Biomaterials 183, 114-127 (2018).
Xiao, Z. et al. Significant Improvement of Acute Complete Spinal Cord Injury Patients Diagnosed by a Combined Criteria Implanted with NeuroRegen Scaffolds and Mesenchymal Stem Cells. Cell Transplant 27, 907-915 (2018).
Davidenko, N. et al. Control of crosslinking for tailoring collagen-based scaffolds stability and mechanics. Acta Biomater 25, 131-142 (2015).
Dikici, S., Claeyssens, F. & MacNeil, S. Decellularised baby spinach leaves and their potential use in tissue engineering applications: Studying and promoting neovascularisation. J Biomater Appl 34, 546-559 (2019).
Peng, W. et al. Recent progress of collagen, chitosan, alginate and other hydrogels in skin repair and wound dressing applications. Int J Biol Macromol 208, 400-408 (2022).
Contessi Negrini, N., Toffoletto, N., Farè, S. & Altomare, L. Plant Tissues as 3D Natural Scaffolds for Adipose, Bone and Tendon Tissue Regeneration. Front Bioeng Biotechnol 8, (2020).
Chen, F. et al. Natural Flammulina velutipes-Based Nerve Guidance Conduit as a Potential Biomaterial for Peripheral Nerve Regeneration: In Vitro and In Vivo Studies. ACS Biomater Sci Eng 7, 3821-3834 (2021).
Lee, J., Jung, H., Park, N., Park, S.-H. & Ju, J. H. Induced Osteogenesis in Plants Decellularized Scaffolds. Sci Rep 9, 20194 (2019).
Venkatesan, J. & Kim, S.-K. Chitosan Composites for Bone Tissue Engineering—An Overview. Mar Drugs 8, 2252-2266 (2010).
Balasundari, R. et al. Nanocoated Botanical Scaffold in Salvage for Human Tissue Regeneration. J Biomater Tissue Eng 2, 330-335 (2012).
Bilirgen, A. C. et al. Plant-Based Scaffolds in Tissue Engineering. ACS Biomater Sci Eng 7, 926-938 (2021).
Modulevsky, D. J., Cuerrier, C. M. & Pelling, A. E. Biocompatibility of Subcutaneously Implanted Plant-Derived Cellulose Biomaterials. PLOS One 11, e0157894 (2016).
Hickey, R. J. & Pelling, A. E. Cellulose Biomaterials for Tissue Engineering. Front Bioeng Biotechnol 7, (2019).
Hickey, R. J., Modulevsky, D. J., Cuerrier, C. M. & Pelling, A. E. Customizing the Shape and Microenvironment Biochemistry of Biocompatible Macroscopic Plant-Derived Cellulose Scaffolds. ACS Biomater Sci Eng 4, 3726-3736 (2018).
Fontana, G. et al. Biofunctionalized Plants as Diverse Biomaterials for Human Cell Culture. Adv Healthc Mater 6, 1601225 (2017).
Couvrette, L. J., Walker, K. L. a., Bui, T. V. & Pelling, a. E. Plant Cellulose As a Substrate for 3D Neural Stem Cell Culture. Bioengineering 10, 1309 (2023).
Rivlin, A. S. & Tator, C. H. Objective clinical assessment of motor function after experimental spinal cord injury in the rat. J Neurosurg 47, 577-581 (1977).
Shah, P. K. et al. Use of quadrupedal step training to re-engage spinal interneuronal networks and improve locomotor function after spinal cord injury. Brain 136, 3362-77 (2013).
Ichiyama, R. M., Gerasimenko, Yu. P., Zhong, H., Roy, R. R. & Edgerton, V. R. Hindlimb stepping movements in complete spinal rats induced by epidural spinal cord stimulation. Neurosci Lett 383, 339-344 (2005).
Hiersemenzel, L. -P., Curt, A. & Dietz, V. From spinal shock to spasticity. Neurology 54, 1574-1582 (2000).
West, S. J., Bannister, K., Dickenson, A. H. & Bennett, D. L. Circuitry and plasticity of the dorsal horn—Toward a better understanding of neuropathic pain. Neuroscience 300, 254-275 (2015).
Hiraga, S., Itokazu, T., Nishibe, M. & Yamashita, T. Neuroplasticity related to chronic pain and its modulation by microglia. Inflamm Regen 42, 15 (2022).
Liu, S. et al. A multi-channel collagen scaffold loaded with neural stem cells for the repair of spinal cord injury. Neural Regen Res 16, 2284-2292 (2021).
Gong, Z. et al. Bioactive Elastic Scaffolds Loaded with Neural Stem Cells Promote Rapid Spinal Cord Regeneration. ACS Biomater Sci Eng 6, 6331-6343 (2020).
Kang, K. N. et al. Regeneration of Completely Transected Spinal Cord Using Scaffold of Poly(D, L-Lactide-co-Glycolide)/Small Intestinal Submucosa Seeded with Rat Bone Marrow Stem Cells. Tissue Eng Part A 17, 2143-2152 (2011).
Li, X. et al. Reactive Astrogliosis: Implications in Spinal Cord Injury Progression and Therapy. Oxid Med Cell Longev 2020, 1-14 (2020).
Singh, A. V. et al. Astrocytes Increase ATP Exocytosis Mediated Calcium Signaling in Response to Microgroove Structures. Sci Rep 5, 7847 (2015).
Schackel, T. et al. Peptides and Astroglia Improve the Regenerative Capacity of Alginate Gels in the Injured Spinal Cord. Tissue Eng Part A 25, 522-537 (2019).
Ge, H. et al. Poly-L-ornithine enhances migration of neural stem/progenitor cells via promoting a-Actinin 4 binding to actin filaments. Sci Rep 6, 37681 (2016).
Takeoka, A. Proprioception: Bottom-up directive for motor recovery after spinal cord injury. Neurosci Res 154, 1-8 (2020). Takeoka, A. & Arber, S. Functional Local Proprioceptive Feedback Circuits Initiate and Maintain Locomotor Recovery after Spinal Cord Injury. Cell Rep 27, 71-85.e3 (2019).
Takeoka, A., Vollenweider, I., Courtine, G. & Arber, S. Muscle Spindle Feedback Directs Locomotor Recovery and Circuit Reorganization after Spinal Cord Injury. Cell 159, 1626-1639 (2014).
Courtine, G. et al. Transformation of nonfunctional spinal circuits into functional states after the loss of brain input. Nat Neurosci 12, 1333-1342 (2009).
Pearson, K. G. Generating the walking gait: role of sensory feedback. in 123-129 (2004). doi: 10.1016/S 0079-6123(03)43012-4.
Musienko, P. E., Bogacheva, I. N. & Gerasimenko, Yu. P. Significance of peripheral feedback in the generation of stepping movements during epidural stimulation of the spinal cord. Neurosci Behav Physiol 37, 181-190 (2007).
Xiong, Y. -J. et al. Poly-L-ornithine blocks the inhibitory effects of fibronectin on oligodendrocyte differentiation and promotes myelin repair. Neural Regen Res 18, 832 (2023).
Koopmans, G. C. et al. Functional Recovery, Serotonergic Sprouting, and Endogenous Progenitor Fates in Response to Delayed Environmental Enrichment after Spinal Cord Injury. J Neurotrauma 29, 514-527 (2012).
Hutson, T. H. et al. Cbp-dependent histone acetylation mediates axon regeneration induced by environmental enrichment in rodent spinal cord injury models. Sci Transl Med 11, (2019).
De Virgiliis, F. et al. Enriched conditioning expands the regenerative ability of sensory neurons after spinal cord injury via neuronal intrinsic redox signaling. Nat Commun 11, 6425 (2020).
Lankhorst, A. J. et al. Effects of Enriched Housing on Functional Recovery After Spinal Cord Contusive Injury in the Adult Rat. J Neurotrauma 18, 203-215 (2001).
Clemenson, G. D., Deng, W. & Gage, F. H. Environmental enrichment and neurogenesis: from mice to humans. Curr Opin Behav Sci 4, 56-62 (2015).
Berrocal, Y. et al. Social and Environmental Enrichment Improves Sensory and Motor Recovery after Severe Contusive Spinal Cord Injury in the Rat. J Neurotrauma 24, 1761-1772 (2007).
Gabriel, A. F., Marcus, M. A. E., Honig, W. M. M., Helgers, N. & Joosten, E. A. J. Environmental housing affects the duration of mechanical allodynia and the spinal astroglial activation in a rat model of chronic inflammatory pain. Brain Res 1276, 83-90 (2009).
Tai, W. L. et al. Additive Effects of Environmental Enrichment and Ketamine on Neuropathic Pain Relief by Reducing Glutamatergic Activation in Spinal Cord Injury in Rats. Front Neurosci 15, (2021).
McDonald, M. W., Hayward, K. S., Rosbergen, I. C. M., Jeffers, M. S. & Corbett, D. Is Environmental Enrichment Ready for Clinical Application in Human Post-stroke Rehabilitation? Front Behav Neurosci 12, (2018).
Neves, L. T. et al. Environmental Enrichment in Stroke Research: an Update. Transl Stroke Res (2023) doi: 10.1007/s12975-023-01132-w.
Rossi, C. et al. Brain-derived neurotrophic factor (BDNF) is required for the enhancement of hippocampal neurogenesis following environmental enrichment. European Journal of Neuroscience 24, 1850-1856 (2006).
Rampon, C. et al. Enrichment induces structural changes and recovery from nonspatial memory deficits in CA1 NMDAR1-knockout mice. Nat Neurosci 3, 238-244 (2000).
Johansson, B. B. & Belichenko, P. V. Neuronal Plasticity and Dendritic Spines: Effect of Environmental Enrichment on Intact and Postischemic Rat Brain. Journal of Cerebral Blood Flow & Metabolism 22, 89-96 (2002).
Zhu, Y. -T. et al. Environmental enrichment combined with fasudil promotes motor function recovery and axonal regeneration after stroke. Neural Regen Res 16, 2512 (2021).
Guo, Y. -S. et al. Effects of enriched environment on microglia and functional white matter recovery in rats with post stroke cognitive impairment. Neurochem Int 154, 105295 (2022).
Maegele, M. et al. Multimodal early onset stimulation combined with enriched environment is associated with reduced CNS lesion volume and enhanced reversal of neuromotor dysfunction after traumatic brain injury in rats. European Journal of Neuroscience 21, 2406-2418 (2005).
BASSO, D. M., BEATTIE, M. S. & BRESNAHAN, J. C. A Sensitive and Reliable Locomotor Rating Scale for Open Field Testing in Rats. J Neurotrauma 12, 1-21 (1995).
Xu, N., Åkesson, E., Holmberg, L. & Sundstrom, E. A sensitive and reliable test instrument to assess swimming in rats with spinal cord injury. Behavioural Brain Research 291, 172-183 (2015).

1. A vascular plant based cellulose scaffold for in-vitro production of neural stem cells.

2. The scaffold of claim 1, wherein the scaffold is obtained by decellularizing a vascular plant or a part thereof.

3. The scaffold of claim 2, wherein the scaffold supports the growth of the neural stem cells.

4. The scaffold of claim 2 or 3, wherein the vascular plant is asparagus or celery.

5. The scaffold of claim 1, wherein the scaffold is coated with a biomolecule, a synthetic biomolecule, a ligand, a protein, an amino acid, an antibody, an extracellular matrix, fibrin, laminin, collagen, fibronectin, poly-l-lysine, a small molecule, a hydrogel, or a matrigel.

6. The scaffold of claim 1, wherein the scaffold is coated with a synthetic biomolecule.

7. The scaffold of claim 1, wherein the scaffold is coated with Poly-L-Ornithine (PLO).

8. The scaffold of claim 1, wherein the scaffold has a porous structure comprising a plurality of vascular bundles with varying diameters and a plurality of parenchymal cells.

9. The scaffold of claim 8, wherein the vascular bundles are interspersed between the parenchymal cells.

10. The scaffold of claim 8, wherein the vascular bundles are unevenly spaced apart from each other and traverse the length of the scaffold.

11. The scaffold of claim 8, wherein the parenchymal cells form the surface of the scaffold.

12. The scaffold of claim 8, wherein the porous structure of the scaffold promotes attachment, proliferation or differentiation of the neural stem cells.

13. The scaffold of claim 8, wherein the porous structure of the scaffold allows the neural stem cells to maintain its differentiation ability.

14. The scaffold of claim 8 wherein the porous structure of the scaffold promotes differentiation of the neural stem cells into neurons, dopaminergic neurons, motor neurons, astrocytes, oligodendrocytes or a combination thereof.

15. The scaffold of claim 8, wherein the porous structure of the scaffold promotes differentiation of the neural stem cells into neurons and astrocytes.

16. The scaffold of claim 7, wherein the poly-L-ornithine coating increases production of the neural stem cells by promoting filopodia formation.

17. The scaffold of claim 8, wherein the porous structure of the scaffold promotes increased production of the neural stem cells by enabling nutrient exchange, oxygen exchange and removal of waste within the scaffold.

18. The scaffold of clam 1, wherein porosity of the scaffold ranges from 10% to 95%.

19. The scaffold of claim 1, wherein the scaffold has an elastic modulus in the range of 1 kPa to 1000 kPa.

20. The scaffold of claim 1, wherein the scaffold is biocompatible.

21. The scaffold of claim 1, wherein the scaffold is biodegradable.

22. The scaffold of claim 1, wherein the surface of the scaffold is modified by a chemical treatment or a physical treatment.

23. The scaffold of claim 22, wherein the modification enhances attachment of the neural stem cells to the surface of the scaffold.

24. The scaffold of claim 22, wherein the surface of the scaffold is treated by functionalizing the surface, gamma irradiation, radical treatment, oxidative treatment, or a combination thereof.

25. The scaffold of claim 24, wherein the functionalization is achieved by providing a functional group that creates a charge on the surface of the scaffold.

26. The scaffold of claim 25, wherein the functional group is a primary amine, a tertiary amine, a quaternary compound, an alcoholic group, a carboxylic acid group, an aldehyde group, a sulfonyl group or a combination thereof.

27. The scaffold of claim 1, wherein the scaffold promotes three-dimensional growth of the neural stem cells as neurospheres on the scaffold.

28. The scaffold of claim 1, wherein the scaffold promotes the cells to grow neurite-like processes.

29. The scaffold of claim 1, wherein the scaffold promotes growth of multiple layers of the neural stem cells.

30. The scaffold of claim 1, wherein the scaffold promotes production of neural cell proteins.

31. The scaffold of any one of claims 1-30, comprising a plurality of linear oriented channels.

32. The scaffold of claim 31, wherein the scaffold promotes growth of neural cells in the plurality of linear oriented channels, preferably growth of endogenous neural cells.

33. The scaffold of any one of claims 30, wherein the scaffold promotes growth and/or regeneration of neuronal sensory tracts.

34. A vascular plant based scaffold according to any one of claims 1-33 for use in regenerative medicine or production of biopharmaceuticals.

35. A vascular plant based scaffold according to any one of claims 1-33 for use in neural tissue engineering.

36. A vascular plant based scaffold according to any one of claims 1-33 for use in the treatment of neural disorders, defects or injuries.

37. A vascular plant based scaffold according to any one of claims 1-33 for use in in-vitro drug production or in-vitro drug testing.

38. A vascular plant based scaffold according to any one of claims 1-33 for use in in-vitro production of neural cell proteins or in-vitro harvesting of neural cell proteins.

39. A vascular plant based scaffold according to any one of claims 1-33 for use in production of neural transplant or neural cell banks.

40. A vascular plant based scaffold according to any one of claims 1-33 for use in production of a neural construct for in-vitro disease modeling.

41. A vascular plant based scaffold according to any one of claims 1-33, for use in treatment of spinal cord injury, preferably in promoting hindlimb motor recovery and/or neural tissue repair in a subject with spinal cord injury.

42. A method of in-vitro production of neural stem cells:
a) seeding a culture medium comprising the neural stem cells in a container on a vascular plant based cellulose scaffold according to any one of claims 1 to 33; and
b) allowing the neural stem cells to attach and grow on the cellulose scaffold for at least 48 hours.

43. The method of claim 42, wherein the culture medium is infused with nutrients and growth factors to facilitate or increase attachment and growth of the neural stem cells to the scaffold.

44. The method of claim 42, wherein the culture medium is DMEM MEM/F12, Aplha MEM, Knockout DMEM/F12, FBS, FCS, Goat Serum, or Horse Serum.

45. The method of claim 42, wherein the culture medium is A:1X KnockOut DMEM/F-12 supplemented with 2% B 27 (cat. 17504044 ThermoFisher), 2 mM GlutaMAX-I (ThermoFisher), B:serum free medium (1X KnockOut D-MEM/F-12 with 2% StemPro Neural Supplement ThermoFisher A1050801), 20 ng/mL bFGF, 20 ng/ml EGF, or 2 mM GlutaMAX-I.

46. The method of claim 42, wherein the method is carried out at a temperature ranging from 32 °C to 42 °C

47. The method of claim 42, wherein the method is carried out at a pH ranging from 6.8 to 7.2.

48. The method of claim 42, wherein the container is a flask, a culture vessel, a bioreactor, a petri dish, a multi-well plate or a glass chamber.

49. The method of claim 42, further comprising a step of coating the container prior to step a) with a natural biomolecule, a synthetic biomolecule, a ligand, a protein, an amino acid, an antibody, an extracellular matrix, fibrin, laminin, collagen, fibronectin, poly-l-lysine, a small molecule, a hydrogel, or a matrigel.

50. The method of claim 49, wherein the container is coated with a natural biomolecule and a synthetic biomolecule.

51. The method of claim 49, wherein the container is coated with poly-L-ornithine and laminin.

52. The method of claim 42, further comprising a pre-processing step prior to step a), wherein the scaffold is pre-processed by:
sterilizing the scaffold with 70% ethanol;
washing the scaffold with a saline solution, a detergent solution, a physiological buffer or a salt solution; or
pre-treating the surface of the scaffold.

53. The method of claim 52, wherein the washing step comprises an incubation step in which the scaffold is incubated in the salt solution overnight.

54. The method of claim 52, wherein the surface is pre-treated by treating the surface chemically, treating the surface physically, functionalizing the surface, succinylating the surface or a combination thereof.

55. The method of claim 54, wherein the surface is pre-treated by treating with a biomolecule, a synthetic biomolecule, a ligand, a protein, an amino acid, an antibody, an extracellular matrix, fibrin, laminin, collagen, fibronectin, poly-l-lysine, a small molecule, a hydrogel, a matrigel or a pharmaceutically acceptable compound.

56. The method of claim 54, wherein the synthetic biomolecule for chemical treatment is poly-L-ornithine.

57. The method of claim 54, wherein the pre-treatment modifies the surface of the scaffold and enhances production of cells by increasing attachment to the surface of the scaffold.

58. The method of claim 54, wherein the surface is functionalized by providing a functional group that creates a charge on the surface of the scaffold.

59. The scaffold of claim 58, wherein the functional group is a primary amine, a tertiary amine, a quaternary compound, an alcoholic group, carboxylic acid, aldehyde, sulfonyl or a combination thereof.

60. The method of claim 42, further comprising a sterilization step prior to step a), wherein the scaffold is sterilized for a pre-determined period of time.

61. The method of claim 60, wherein the sterilization step is carried out by autoclaving, treatment with 70% ethanol, gamma irradiation, or treatment with ethylene oxide.

62. The method of claim 60, wherein the sterilization step is carried out for 20 minutes to several days.

63. The method of claim 42, wherein the production of neural stem cells is increased by modulating stiffness of the scaffold.

64. The method of claim 42, wherein the production of neural stem cells is increased modulating anisotropy of the scaffold.

65. The method of claim 52, further comprising a quantification step to quantify the production of the neural stem cells.

66. The method of claim 65, wherein the quantification step is carried out by a biomolecule quantification assay, cell quantification assay, cell viability assay, image analysis, immunohistochemistry or electrophysiology.

67. The method of claim 66, wherein the quantification step is followed by an optimization step to optimize the growth conditions in the container, to optimize the quantity of scaffold, to optimize the size of the scaffold, to optimize the structure of the scaffold or a combination thereof.

68. The method of claim 42, further comprising an incubation step wherein the scaffold and the neural stem cells are incubated at a temperature ranging from 30 °C to 45 °C

69. The method of claim 68, wherein the scaffold and the neural stem cells are incubated with CO₂.

70. The method of claim 69, wherein the scaffold and the neural stem cells are incubated at 37 °C with 5% CO₂.

71. The method of claim 42, wherein the neural stem cells differentiate to neurons, astrocytes, oligodendrocytes, or a combination thereof after attaching to the scaffold.

72. The method of claim 42, wherein the neural stem cells differentiate to neurons and astrocytes after attaching to the scaffold.

73. The method of claim 71 and 72, wherein the neural stem cell differentiation is determined by an immunostaining assay, cell imaging, confocal microscopy, flow cytometry, or electrophysiology.

74. The method of claim 56, wherein the poly-L-ornithine coating increases production of the neural stem cells by promoting filopodia formation.
